(19) United States
(12) Patent Application Publication
(10) Pub. No.: US 2008/0234335 A1
(43) Pub. Date: Sep. 25, 2008

(54) Cyclohexene Compounds

(76) Inventors: Gerard Martin Paul Giblin, Essex (GB); Adrian Hall, Essex (GB); David Nigel Hurst, Essex (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(21) Appl. No.: 10/574,886

(22) PCT Filed: Oct. 6, 2004
(86) PCT No.: PCT/EP2004/011365

§ 371 Date: May 22, 2006

(30) Foreign Application Priority Data
Oct. 8, 2003 (GB) 0323584.3

(51) Int. Cl. A61K 31/44; C07D 213/79; A61P 29/00

(52) U.S. Cl. 514/354; 546/326

(57) ABSTRACT

Compounds of formula (I) wherein A, B, Z, R¹, R^{2a}, R^{2b}, R⁸, R⁹ and R^{x} are as defined in the specification, a process for the preparation of such compounds, pharmaceutical compositions comprising such compounds and the use of such compounds in medicine.

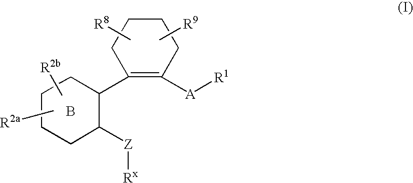

This invention relates to cyclohexene compounds, to processes for their preparation, to pharmaceutical compositions containing them and to their use in medicine, in particular their use in the treatment of conditions mediated by the action of PGE₂ at EP₁ receptors.

The EP₁ receptor is a 7-transmembrane receptor and its natural ligand is the prostaglandin PGE₂. PGE₂ also has affinity for the other EP receptors (types EP₂, EP₃ and EP₄). The EP₁ receptor is associated with smooth muscle contraction, pain (in particular inflammatory, neuropathic and visceral), inflammation, allergic activities, renal regulation and gastric or enteric mucus secretion. We have now found a novel group of compounds which bind with high affinity to the EP₁ receptor.

A number of review articles describe the characterization and therapeutic relevance of the prostanoid receptors as well as the most commonly used selective agonists and antagonists: Eicosanoids; From Biotechnology to Therapeutic Applications, Folco, Samuelsson, Maclouf, and Velo eds, Plenum Press, New York, 1996, chap. 14, 137-154 and Journal of Lipid Mediators and Cell Signalling, 1996, 14, 83-87 and Prostanoid Receptors, Structure, Properties and Function, S Narumiya et al, Physiological Reviews 1999, 79(4), 1193-126. An article from The British Journal of Pharmacology, 1994, 112, 735-740 suggests that Prostaglandin E₂ (PGE₂) exerts allodynia through the EP₁ receptor subtype and hyperalgesia through EP₂ and EP₃ receptors in the mouse spinal cord. Furthermore an article from The Journal of Clinical Investigation, 2001, 107 (3), 325 shows that in the EP₁ knock-out mouse pain-sensitivity responses are reduced by approximately 50%. Two papers from Anesthesia and Analgesia have shown that (2001, 93, 1012-7) an EP₁ receptor antagonist (ONO-8711) reduces hyperalgesia and allodynia in a rat model of chronic constriction injury, and that (2001, 92, 233-238) the same antagonist inhibits mechanical hyperalgesia in a rodent model of post-operative pain. S. Sarkar et al in Gastroenterology, 2003, 124(1), 18-25 demonstrate the efficacy of EP₁ receptor antagonists in the treatment of visceral pain in a human model of hypersensitivity. Thus, selective prostaglandin ligands, agonists or antagonists, depending on which prostaglandin E receptor subtype is being considered, have anti-inflammatory, antipyretic and analgesic properties similar to a conventional non-steroidal anti-inflammatory drug, and in addition, inhibit hormone-induced uterine contractions and have anti-cancer effects. These compounds have a diminished ability to induce some of the mechanism-based side effects of NSAIDs which are indiscriminate cyclooxygenase inhibitors. In particular, the compounds have a reduced potential for gastrointestinal toxicity, a reduced potential for renal side effects, a reduced effect on bleeding times and a lessened ability to induce asthma attacks in aspirin-sensitive asthmatic subjects. Moreover, by sparing potentially beneficial prostaglandin pathways, these agents may have enhanced efficacy over NSAIDS and/or COX-2 inhibitors.

In The American Physiological Society (1994, 267, R289R-294), studies suggest that PGE₂-induced hyperthermia in the rat is mediated predominantly through the EP₁ receptor.

WO 96/06822 (Mar. 7, 1996), WO 96/11902 (Apr. 25, 1996), EP 752421-A1 (Jan. 8, 1997), WO 01/19814 (22 Mar. 2001), WO 03/084917 (16 Oct. 2003), WO 03/101959 (11 Dec. 2003) and WO 2004/039753 (13 May 2004) disclose compounds as being useful in the treatment of prostaglandin mediated diseases.

It is now suggested that a novel group of cyclohexene derivatives surprisingly are selective for the EP₁ receptor over the EP₃ receptor, and are therefore indicated to be useful in treating conditions mediated by the action of PGE₂ at EP₁ receptors. Such conditions include pain, or inflammatory, immunological, bone, neurodegenerative or renal disorders.

Accordingly the present invention provides cyclohexene compounds of formula (I):

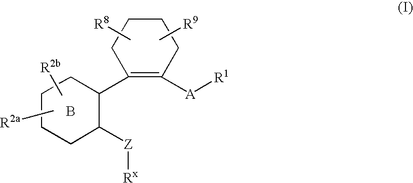

wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl,
SO₂NR⁵R⁶, NR⁵CONR⁵R⁶, COalkyl, 2H-tetrazol-5-ylmethyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2, optionally substituted alkenyl or optionally substituted alkynyl: or R^{x} represents optionally substituted CQ^{a}Q^{b} heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} each independently selected from hydrogen and CH₃; and
when A is a 6-membered ring the R¹ substituent and cyclohexene ring are attached to carbon atoms 1,2-, 1,3- or 1,4-relative to each other, and when A is a five-membered ring or bicyclic heterocyclyl group the R¹ substituent and cyclohexene ring are attached to substitutable carbon atoms 1,2- or 1,3-relative to each other, and derivatives thereof.

Suitably A includes optionally substituted pyridyl.

Optional substituents for A include up to four substituents, preferably 0 or 1 substituent, independently selected from for example halogen, NR⁵R⁶, NR⁵COC₁₋₆alkyl, NR⁵SO₂C₁₋₆alkyl, OR⁵, optionally substituted C₁₋₄alkyl, wherein R⁵ and R⁶ are as defined above.

In one aspect B is phenyl.

Preferably Z is O.

Suitably R¹ includes CO₂H

Particular examples of R^{2a} and R^{2b} include hydrogen, halo, and optionally substituted C₁₋₆alkyl, e.g. CF₃. Preferably R^{2a} and R^{2b} are independently selected from hydrogen, Cl, Br and CF₃.

Preferably R^{2a} is hydrogen. Preferably R^{2b} is positioned 1,4-relative to the Z substituent and 1,3-relative to the cyclohexene ring.

Suitably R⁴ includes hydrogen or C₁₋₄alkyl.

Suitably R⁵ includes hydrogen or C₁₋₄alkyl.

Suitably R⁶ includes hydrogen or C₁₋₄alkyl.

Suitably R⁷ includes hydrogen or C₁₋₄alkyl.

Suitably R⁸ and R⁹ are each hydrogen.

Suitably R^{x} when an optionally substituted alkyl group includes C₁₋₈alkyl.

Suitably R^{x} include CH₂phenyl optionally substituted by one, two or three, preferably one or two substituents, selected from halogen, e.g. Cl and F, and CF₃.

An example of Q^{a} is hydrogen.

An example of Q^{b} is hydrogen.

A certain group of compounds of formula (I) are compounds of formula (Ia):

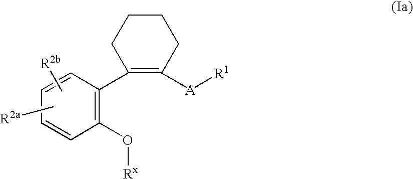

wherein:
A is a pyridyl ring;

### R¹ is CO₂H;

R^{2a} and R^{2b} are independently selected from hydrogen, halo, optionally and substituted C₁₋₄alkyl e.g. CF₃.
R^{x} is optionally substituted CH₂-phenyl;
or derivatives thereof.

Preferably R^{2a} is hydrogen.

Suitably R^{2b} represents hydrogen, halogen e.g. Cl or F, or CF₃.

Preferably R^{2b} is positioned 1,4-relative to the OR^{x} substituent and 1,3-relative to the cyclohexene ring.

Preferably R^{x} is CH₂-phenyl optionally substituted by one, two or three substituents selected from halogen e.g. Cl or F, and CF₃.

Compounds of formula (I) include:
6-[2-(5-chloro-2-{[(4-fluorophenyl)methyl]oxy}phenyl)-1-cyclohexen-1-yl]-2-pyridinecarboxylic acid;
6-[2-(5-chloro-2-{[(2,4-difluorophenyl)methyl]oxy}phenyl)-1-cyclohexen-1-yl]-2-pyridinecarboxylic acid; and
6-[2-(5-chloro-2-{[(2,4-difluorophenyl)methyl]oxy}phenyl)-1-cyclohexen-1-yl]-2-pyridinecarboxylic acid;
6-{2-[2-{[(4-fluorophenyl)methyl]oxy}-5-(trifluoromethyl)phenyl]-1-cyclohexen-1-yl}-2-pyridinecarboxylic acid;
6-{2-[2-{[(2,4-difluorophenyl)methyl]oxy}-5-(trifluoromethyl)phenyl]-1-cyclohexen-1-yl}-2-pyridinecarboxylic acid;
6-[2-(5-(trifluoromethyl)-2-{[(2,4,5-trifluorophenyl)methyl]oxy}phenyl)-1-cyclohexen-1-yl]-2-pyridinecarboxylic acid;
6-{2-[2-{[(4-chloro-2-fluorophenyl)methyl]oxy}-5-(trifluoromethyl)phenyl]-1-cyclohexen-1-yl}-2-pyridinecarboxylic acid;
6-[2-(5-(trifluoromethyl)-2-{[(2,4,6-trifluorophenyl)methyl]oxy}phenyl)-1-cyclohexen-1-yl]-2-pyridinecarboxylic acid;
6-{2-[2{[(2-chlorophenyl)methyl]oxy}-5-(trifluoromethyl)phenyl]-1-cyclohexen-1-yl}2-pyridinecarboxylic acid;
6-{2-[2-{[(3,4-difluorophenyl)methyl]oxy}-5-(trifluoromethyl)phenyl]-1-cyclohexen-1-yl}-2-pyridinecarboxylic acid;
6-{2-[2{[(2-chloro-4-fluorophenyl)methyl]oxy}-5-(trifluoromethyl)phenyl]-1-cyclohexen-1-yl}-2-pyridinecarboxylic acid;
6-{2-[2-{[(4-chlorophenyl)methyl]oxy}-5-(trifluoromethyl)phenyl]-1-cyclohexen-1-yl}-2-pyridinecarboxylic acid;
6-{2-[2{[(2-fluorophenyl)methyl]oxy}-5-(trifluoromethyl)phenyl]-1-cyclohexen-1-yl}-2-pyridinecarboxylic acid;
6-{2-[2-[(phenylmethyl)oxy]-5-(trifluoromethyl)phenyl]-1-cyclohexen-1-yl}-2-pyridinecarboxylic acid;
5-{2-[2-{[(2-fluorophenyl)methyl]oxy}-5-(trifluoromethyl)phenyl]-1-cyclohexen-1-yl}-3-pyridinecarboxylic acid;
5-{2-[2-{[(2,4-difluorophenyl)methyl]oxy}-5-(trifluoromethyl)phenyl]-1-cyclohexen-1-yl}-3-pyridinecarboxylic acid;
5-[2-(5-(trifluoromethyl)-2-{[(2,4,6-trifluorophenyl)methyl]oxy}phenyl)-1-cyclohexen-1-yl]-3-pyridinecarboxylic acid;
5-{2-[2-{[(4-fluorophenyl)methyl]oxy}-5-(trifluoromethyl)phenyl]-1-cyclohexen-1-yl}-3-pyridinecarboxylic acid;
5-[2-(5-(trifluoromethyl)-2-{[(2,3,4-trifluorophenyl)methyl]oxy}phenyl)-1-cyclohexen-1-yl]-3-pyridinecarboxylic acid;
5-[2-(5-(trifluoromethyl)-2{[(2,4,5-trifluorophenyl)methyl]oxy}phenyl)-1-cyclohexen-1-yl]-3-pyridinecarboxylic acid;
5-{2-[2-{[(2-chloro-4-fluorophenyl)methyl]oxy}-5-(trifluoromethyl)phenyl]-1-cyclohexen-1-yl}-3-pyridinecarboxylic acid;
5-{2-[2-{[(4-chloro-2-fluorophenyl)methyl]oxy}-5-(trifluoromethyl)phenyl]-1-cyclohexen-1-yl}-3-pyridinecarboxylic acid;
5-{2-[2-[(phenylmethyl)oxy]-5-trifluoromethyl)phenyl]-1-cyclohexen-1-yl}-3-pyridinecarboxylic acid;
6-[2-(5-chloro-2-{[(2,4,5-trifluorophenyl)methyl]oxy}phenyl)-1-cyclohexen-1-yl]-2-pyridinecarboxylic acid;
6-[2-(5-chloro-2-{[(2-fluorophenyl)methyl]oxy}phenyl)-1-cyclohexen-1-yl]-2-pyridinecarboxylic acid;
6-[2-(5-chloro-2-{[(2,4,6-trifluorophenyl)methyl]oxy}phenyl)-1-cyclohexen-1-yl]-2-pyridinecarboxylic acid;
6-[2-(5-chloro-2-{[(2-chloro-4-fluorophenyl)methyl]oxy}phenyl)-1-cyclohexen-1-yl]-2-pyridinecarboxylic acid;
6-[2-(5-chloro-2-{[(3,4,5-trifluorophenyl)methyl]oxy}phenyl)-cyclohexen-1-yl]-2-pyridinecarboxylic acid;
6-[2-(5-chloro-2-{[(3,4-difluorophenyl)methyl]oxy}phenyl)-1-cyclohexen-1-yl]-2-pyridinecarboxylic acid;
6-[2-(5-chloro-2-{[(4-chloro-2-fluorophenyl)methyl]oxy}phenyl)-1-cyclohexen-1-yl]-2-pyridinecarboxylic acid;
6-[2-(5-chloro-2{[(4-chlorophenyl)methyl]oxy}phenyl)-1-cyclohexen-1-yl]-2-pyridinecarboxylic acid;
5-(2-{5-chloro-2-[(phenylmethyl)oxy]phenyl}-1-cyclohexen-1-yl)-3-pyridinecarboxylate
5-[2-(5-chloro-2-{[(2-fluorophenyl)methyl]oxy}phenyl)-1-cyclohexen-1-yl]-3-pyridinecarboxylic acid;
5-[2-(5-chloro-2-{[(4-fluorophenyl)methyl]oxy}phenyl)-1-cyclohexen-1-yl]-3-pyridinecarboxylic acid;
5-[2-(5-chloro-2-{[(2,4-difluorophenyl)methyl]oxy}phenyl)-1-cyclohexen-1-yl]-3-pyridinecarboxylic acid;
5-[2-(5-chloro-2-{[(2,4,5-trifluorophenyl)methyl]oxy}phenyl)-1-cyclohexen-1-yl]-3-pyridinecarboxylic acid;
5-[2-(5-chloro-2-{[(2,3,4-trifluorophenyl)methyl]oxy}phenyl)-1-cyclohexen-1-yl]-3-pyridinecarboxylic acid;
5-[2-(5-chloro-2-{[(2-chloro-4-fluorophenyl)methyl]oxy}phenyl)-1-cyclohexen-1-yl]-3-pyridinecarboxylic acid;
6-(2-{5-bromo-2-[(phenylmethyl)oxy]phenyl}-1-cyclohexen-1-yl)-2-pyridinecarboxylic acid;
6-[2-(5-bromo-2-{[(2-fluorophenyl)methyl]oxy}phenyl)-1-cyclohexen-1-yl]-2-pyridinecarboxylic acid;
6-[2-(5-bromo-2-{[(4-fluorophenyl)methyl]oxy}phenyl)-1-cyclohexen-1-yl]-2-pyridinecarboxylic acid;
6-[2-(5-bromo-2-{[(2,4-difluorophenyl)methyl]oxy}phenyl)-1-cyclohexen-1-yl]-2-pyridinecarboxylic acid;
6-[2-(5-bromo-2-{[(3,4-difluorophenyl)methyl]oxy}phenyl)-1-cyclohexen-1-yl]-2-pyridinecarboxylic acid;
6-[2-(5-bromo-2{[(2,3,4-trifluorophenyl)methyl]oxy}phenyl)-1-cyclohexen-1-yl]-2-pyridinecarboxylic acid;
6-[2-(5-bromo-2-{[(2,4,5-trifluorophenyl)methyl]oxy}phenyl)-1-cyclohexen-1-yl]-2-pyridinecarboxylic acid;
6-[2-(5-bromo-2-{[(2,4,6-trifluorophenyl)methyl]oxy}phenyl)-1-cyclohexen-1-yl]-2-pyridinecarboxylic acid;
6-[2-(5-bromo-2-{[(2-chloro-4-fluorophenyl)methyl]oxy}phenyl)-1-cyclohexen-1-yl]-2-pyridinecarboxylic acid; and
3-[2-(5-chloro-2-{[(2,4-difluorophenyl)methyl]oxy}phenyl)-1-cyclohexen-1-yl]-2-pyridinecarboxylic acid;
   and derivatives thereof.

The compounds of the invention are selective for EP₁ over EP₃. The compounds of the examples are at least 20 fold selective. Preferred compounds are at least 100 fold selective for EP₁ over EP₃.

Derivatives of the compounds of formula (I) include pharmaceutically acceptable derivatives.

The invention is described using the following definitions unless otherwise indicated.

The term “pharmaceutically acceptable derivative” means any pharmaceutically acceptable salt, solvate, ester, or solvate of salt or ester of the compounds of formula (I), or any other compound which upon administration to the recipient is capable of providing (directly or indirectly) a compound of formula (I).

It will be appreciated by those skilled in the art that the compounds of formula (I) may be modified to provide pharmaceutically acceptable derivatives thereof at any of the functional groups in the compounds, and that the compounds of formula (I) may be derivatised at more than one position.

It will be appreciated that, for pharmaceutical use, the salts referred to above will be pharmaceutically acceptable salts, but other salts may find use, for example in the preparation of compounds of formula (I) and the pharmaceutically acceptable salts thereof.

Pharmaceutically acceptable salts include those described by Berge, Bighley and Monkhouse, J. Pharm. Sci., 1977, 66, 1-19. The term “pharmaceutically acceptable salts” refers to salts prepared from pharmaceutically acceptable bases including inorganic bases and organic bases. Salts derived from inorganic bases include aluminum, ammonium, calcium, copper, ferric, ferrous, lithium, magnesium, manganic salts, manganous, potassium, sodium, zinc, and the like. A particular salt is the sodium salt. Salts derived from pharmaceutically acceptable organic bases include salts of primary, secondary, and tertiary amines; substituted amines including naturally occurring substituted amines; and cyclic amines. Particular pharmaceutically acceptable organic bases include arginine, betaine, caffeine, choline, N,N′-dibenzylethylenediamine, diethylamine, 2-diethylaminoethanol, 2-dimethylaminoethanol, ethanolamine, ethylenediamine, N-ethyl-morpholine, N-ethylpiperidine, glucamine, glucosamine, histidine, hydrabamine, isopropylamine, lysine, methylglucamine, morpholine, piperazine, piperidine, procaine, purines, theobromine, triethylamine, trimethylamine, tripropyl amine, tromethamine, and the like. Salts may also be formed from basic ion exchange resins, for example polyamine resins. When the compound of the present invention is basic, salts may be prepared from pharmaceutically acceptable acids, including inorganic and organic acids. Such acids include acetic, benzenesulfonic, benzoic, camphorsulfonic, citric, ethanesulfonic, ethanedisulfonic, fumaric, gluconic, glutamic, hydrobromic, hydrochloric, isethionic, lactic, maleic, malic, mandelic, methanesulfonic, mucic, pamoic, pantothenic, phosphoric, propionic, succinic, sulfuric, tartaric, p-toluenesulfonic acid, and the like.

The compounds of formula (I) may be prepared in crystalline or non-crystalline form, and if crystalline, may be optionally hydrated or solvated. This invention includes in its scope stoichiometric hydrates as well as compounds containing variable amounts of water.

Suitable solvates include pharmaceutically acceptable solvates, such as hydrates.

Solvates include stoichiometric solvates and non-stoichiometric solvates.

The terms “halogen” or “halo” are used to represent fluorine, chlorine, bromine or iodine.

The term “alkyl” as a group or part of a group means a straight, branched or cyclic chain alkyl group or combinations thereof. Unless hereinbefore defined, examples of alkyl include C₁₋₆alkyl, for example methyl, ethyl, n-propyl, iso-propyl, n-butyl, sec-butyl, iso-butyl, t-butyl, pentyl, hexyl, 1,1-dimethylethyl, cyclopentyl or cyclohexyl or combinations thereof such as cyclohexylmethyl and cyclopentylmethyl.

The term “alkoxy” as a group or as part of a group means a straight, branched or cyclic chain alkoxy group. Unless hereinbefore defined “alkoxy” includes C₁₋₆alkoxy, e.g. methoxy, ethoxy, n-propoxy, iso-propoxy, n-butoxy, sec-butoxy, iso-butoxy, t-butoxy, pentoxy, hexyloxy, cyclopentoxy or cyclohexyloxy. In one aspect “alkoxy” is C₁₋₆ alkoxy.

The term “alkenyl” means linear or branched structures and combinations thereof, of the indicated number of carbon atoms, having at least one carbon-to-carbon double bond, wherein hydrogen may be replaced by an additional carbon to carbon double bond. In one aspect “alkenyl” is C₂₋₆alkenyl, for example ethenyl, propenyl, 1-methylethenyl, butenyl and the like.

The term “alkynyl” means linear or branched structures and combinations thereof, of the indicated number of carbon atoms, having at least one carbon-to-carbon triple bond. C₂₋₆alkynyl, for example, includes ethynyl, propynyl, butynyl and the like.

The term “heterocyclyl” as a group or as part of a group means an aromatic or non-aromatic five or six membered ring which contains from 1 to 4 heteroatoms selected from nitrogen, oxygen or sulfur and is unsubstituted or substituted by, for example, up to three substituents. Examples of 5-membered heterocyclyl groups include furyl, dioxalanyl, pyrrolyl, oxazolyl, thiazolyl, imidazolyl, oxadiazolyl, thiadiazolyl, triazolyl, triazinyl, isothiazolyl, isoxazolyl, thiophenyl, pyrazolyl or tetrazolyl. Examples of 6-membered heterocycyl groups are pyridyl, pyridazinyl, pyrimidinyl, pyrazinyl, triazinyl or tetrazinyl.

The term “aryl” as a group or part of a group means a 5- or 6-membered aromatic ring, for example phenyl, or a 7 to 12 membered bicyclic ring system where at least one of the rings is aromatic, for example naphthyl. An aryl group may be optionally substituted by one or more substituents, for example up to 4, 3 or 2 substituents. Preferably the aryl group is phenyl.

The term “heteroaryl” as a group or as part of a group means a monocyclic five or six membered aromatic ring, or a fused bicyclic aromatic ring system comprising two of such monocyclic five or six membered aromatic rings. These heteroaryl rings contain one or more heteroatoms selected from nitrogen, oxygen or sulfur, where N-oxides, sulfur oxides and sulfur dioxides are permissible heteroatom substitutions. A heteroaryl group may be optionally substituted by one or more substituents, for example up to 3 or up to 2 substituents. Examples of “heteroaryl” used herein include furyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyrimidinyl, quinolinyl, isoquinolinyl, benzofuryl, benzothienyl, indolyl, and indazolyl.

The term “bicyclic heterocyclyl” when used herein means a fused bicyclic aromatic or non-aromatic bicyclic heterocyclyl ring system comprising up to four, preferably one or two, heteroatoms each selected from oxygen, nitrogen and sulphur. Each ring may have from 4 to 7, preferably 5 or 6, ring atoms. A bicyclic heteroaromatic ring system may include a carbocyclic ring. Examples of bicyclic heterocyclyl groups include quinolinyl, isoquinolinyl, quinoxalinyl, quinazolinyl, pyridopyrazinyl, benzoxazolyl, benzothiophenyl, benzimidazolyl, benzothiazolyl, benzoxadiazolyl, benzthiadiazolyl, indolyl, benztriazolyl or naphthyridinyl.

When the heteroatom nitrogen replaces a carbon atom in an alkyl group, or when nitrogen is present in a heteroaryl, heterocyclyl or bicyclic heterocyclyl group, the nitrogen atom will, where appropriate, be substituted by one or two substituents selected from hydrogen and C₁₋₆-alkyl, preferably hydrogen and C₁₋₆alkyl, more preferably hydrogen.

Optional substituents for alkyl, alkenyl or alkynyl groups unless hereinbefore defined include OH, CO₂H, CO₂C₁₋₆alkyl, NHC₁₋₆alkyl, NH₂, (O), OC₁₋₆alkyl, phenyl or halo e.g. Cl, Br or F. An alkyl, alkenyl or alkynyl group may be substituted by one or more optional substituents, for example up to 5, 4, 3, 2 or 1 optional substituents. Particular substituted alkyl groups include those substituted by one or more fluorine atoms, up to per-fluorination, e.g. CH₂F, CHF₂, CF₃, C₂F₅, CH₂CF₃, and CH₂CH₂CF₃.

Optional substituents for alkoxy groups unless hereinbefore defined include OH, and halo e.g. Cl, Br or F. An alkoxy group may be substituted by one or more optional substituents, for example up to 5, 4, 3, or 2 optional substituents.

Unless otherwise defined, optional substituents for aryl, heteroaryl or heterocyclyl moieties as a group or part of a group are selected from C₁₋₆alkyl, C₁₋₆alkoxy and halogen.

Compounds of formula (I) can be prepared as set forth in the following schemes and in the examples. The following processes form another aspect of the present invention.

For example, compounds of formula (I) may be prepared by the general route below:

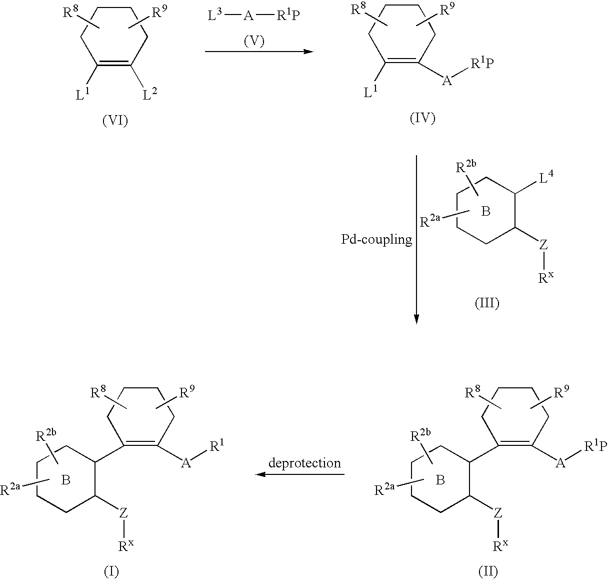

wherein L¹ and L² each represent a leaving group for example halo, e.g. bromo or chloro, or triflate; L³ and L⁴ each represent an activating group, for example boronic acid; P is an optional protecting group; and A, B, R¹, R^{2a}, R^{2b}, R⁸, R⁹, Z and R^{x} are as defined for compounds of formula (I). L¹ can be converted to L^{1a}, and L² can be converted to L^{2a} wherein L^{1a} and L^{2a} each represent an activating group for example a boronic acid, and in this situation L³ and L⁴ can each be halo or triflate.

When R¹ is CO₂H examples of P include methyl, ethyl or benzyl esters.

Suitable reaction conditions for the deprotection of a compound of formula (II) include heating in ethanolic sodium hydroxide solution.

Suitable reaction conditions for the reaction of a compound of formula (VI) with a boronic acid of formula (V, L³ is —B(OH)₂), or a compound of formula (IV) with a boronic acid of formula (III, L⁴ is —B(OH)₂) include heating with tetrakis(triphenylphosphine)palladium (0) and an inorganic base, for example potassium carbonate, in a solvent, e.g. ethylene glycol dimethyl ether (DME), toluene and ethanol, preferably in a ratio of 1:1.

Accordingly the present invention also provides a process for the preparation of a cyclohexene compound of formula (I) or a derivative thereof:

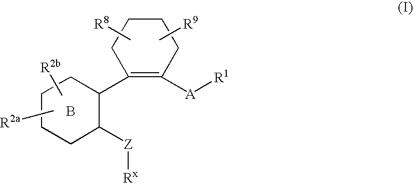

wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2, optionally substituted alkenyl or optionally substituted alkynyl: or R^{x} represents optionally substituted CQ^{a}Q^{b}heterocyclyl, optionally substituted CQ^{a}Q^{b}-bicyclic heterocycyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} each independently selected from hydrogen and CH₃; and
when A is a 6 membered ring the R¹ substituent and cyclohexene ring are attached to carbon atoms 1,2-, 1,3- or 1,4-relative to each other, and when A is a five-membered ring or bicyclic heterocyclyl group the R¹ substituent and cyclohexene ring are attached to substitutable carbon atoms 1,2- or 1,3-relative to each other, comprising:
reacting a compound of formula (IV):

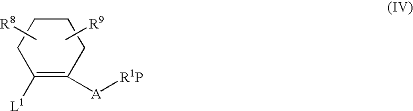

wherein R⁸, R⁹, A, and R¹ are as hereinbefore defined above for a compound of formula (I), L¹ is a leaving group and P is an optional protecting group;
with a compound of formula (III):

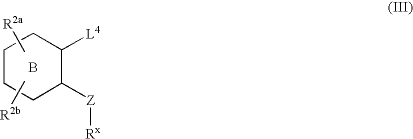

wherein R^{2a}, R^{2b}, B, Z, and R^{x} are as hereinbefore defined above for a compound of formula (I) and L⁴ is an activating group;
and where required converting:
one group R^{x} to another group R^{x};
and where required carrying out the following optional steps in any order:
effecting deprotection; and/or
converting one group R¹ to another group R¹; and/or
forming a derivative of the compound of formula (I) so formed.

Alternatively compounds of formula (I) may be prepared according to the route described below:

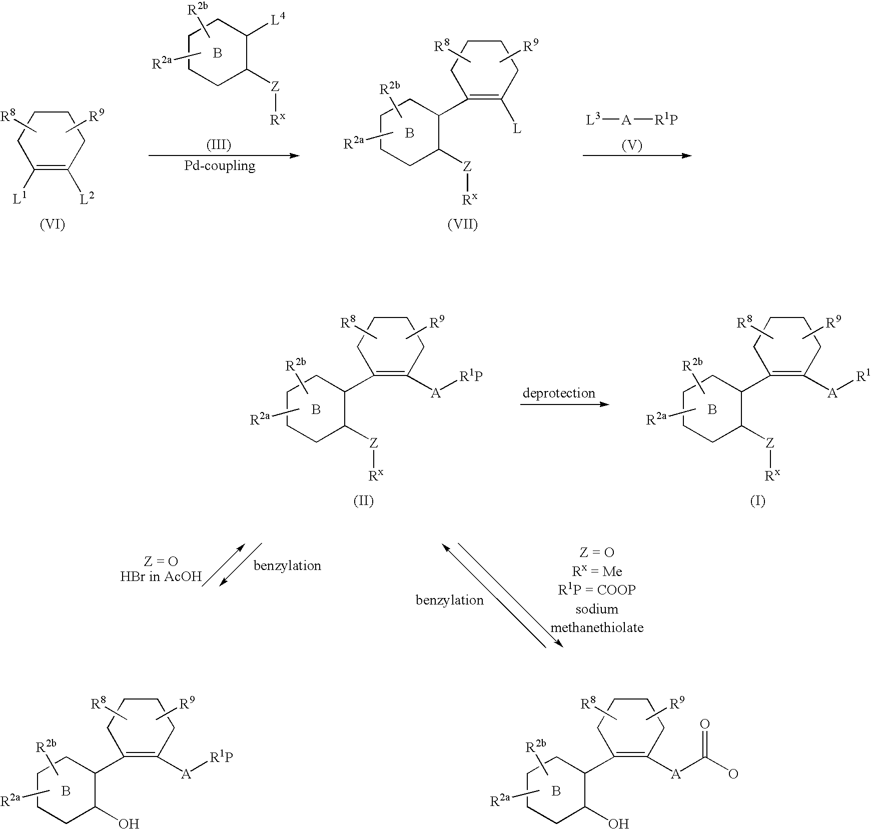

wherein L¹, L², L³, L⁴ and P are as defined above, and A, B, R¹, R^{2a}, R^{2b}, R⁸, R⁹, Z, and R^{x} are as defined for compounds of formula (I). L¹ can be converted to L^{1a}, and L² can be converted to L^{2a} wherein L₁ₐ and L^{2a} each represent an activating group, for example a boronic acid, and in this situation L³ and L⁴ can be halo or triflate.

Accordingly the present invention also provides a process for the preparation of a cyclohexene compound of formula (I) or a derivative thereof:

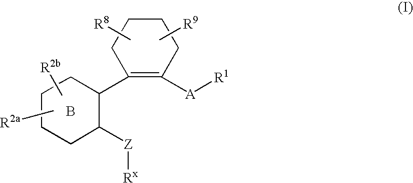

wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ,
wherein n is 0, 1 or 2, optionally substituted alkenyl or optionally substituted alkynyl: or R^{x} represents optionally substituted CQ^{a}Q^{b}heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b}aryl, optionally substituted CQ^{a}Q^{b}heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} each independently selected from hydrogen and CH₃; and
when A is a 6-membered ring the R¹ substituent and cyclohexene ring are attached to carbon atoms 1,2-, 1,3- or 1,4-relative to each other, and when A is a five-membered ring or bicyclic heterocyclyl group the R¹ substituent and cyclohexene ring are attached to substitutable carbon atoms 1,2- or 1,3-relative to each other, comprising:
reacting a compound of formula (VII):

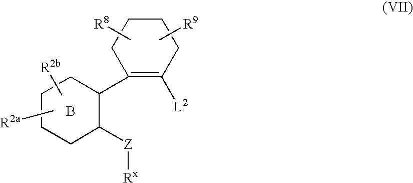

wherein R^{2a}, R^{2b}, R⁸, R⁹, B, and R^{x} are as hereinbefore defined above for a compound of formula (I), and L² is a leaving group;
with a compound of formula (V):

L³-A-R¹P (V)

wherein R¹, and A are as hereinbefore defined above for a compound of formula (I); L³ is an activating group and P is an optional protecting group;
and where required converting:
one group R^{x} to another group R^{x};
and where required carrying out the following optional steps in any order:
effecting deprotection; and/or
converting one group R¹ to another group R¹; and/or
forming a derivative of the compound of formula (I) so formed.

It will be appreciated that certain substituents in intermediates and compounds of formula (I) may be converted to other substituents by conventional methods known to those skilled in the art.

A group R¹ may be converted to another group R¹ by use of conventional organic transformations known to those skilled in the art. For example R¹=CO₂H may be converted to an amide, e.g. CONHCQ^{a}Q^{b}aryl or CONHCQ^{a}Q^{b}heteroaryl wherein Q^{a} and Q^{b} are selected from hydrogen and CH₃, by conventional methods for the preparation of amides as described in, for example, Richard Larock, Comprehensive Organic Transformations, 2nd edition, Wiley-VCH, ISBN 0-471-19031-4.

Cyclohexene derivatives of formula (VI), boronic acids of formula (III) and (V), and tetrakis(triphenylphosphine)palladium (0) are commercially available, or readily prepared by methods known to those skilled in the art.

The preparation and reactions of boronic acids of formula (III) and formula (V) is reviewed in Suzuki et al, Synth. Commun., 1981, 11, 513; Martin et al, Acta. Chim. Scand., 1993, 47, 221; and Miyaura et al, Chem. Rev., 1995, 95, 2457. For example, 2-benzyloxy-5-chlorophenylboronic acid may be prepared from 2-benzyloxy-5-chloro-iodobenzene. 2-Benzyloxy-5-chloro-iodobenzene may be prepared from 4-chloro-2-iodoanisole by demethylation followed by benzylation according to known methods.

Certain substituents in any of the reaction intermediates and compounds of formula (I) may be converted to other substituents by conventional methods known to those skilled in the art. Examples of substituents which may be converted include one group R^{x} to another group R^{x}; and one substituent on a group A to another substituent on a group A. Examples of such transformations include the reduction of a nitro group to give an amino group; alkylation and amidation of amino groups; hydrolysis of esters, alkylation of hydroxy and amino groups; and amidation and esterification of carboxylic acids. Such transformations are well known to those skilled in the art and are described in for example, Richard Larock, Comprehensive Organic Transformations, 2nd edition, Wiley-VCH, ISBN 0-471-19031-4.

For example, when R^{x} is p-methoxybenzyl, cleavage of the ether to give the phenol or pyridinol is carried out using, for example, using acid e.g. HCl/dioxane, HBr/acetic acid or using sodium methanethiolate. When R^{x} is methyl, cleavage of the ether to give the phenol is carried out using, for example, sodium methanethiolate. Cleavage of the ether to give a pyridinol is carried out in the presence of, for example, trifluoroacetic acid. Conversion to another R^{x} group, for example a substituted benzyl group, may be effected by reaction of the phenol or pyridinol with a suitable substituted benzyl bromide. The skilled person will appreciate that conversion of the protecting group P to another protecting group P may also occur under the reaction conditions used. When R^{x} is benzyl, cleavage of the ether to give the phenol or pyridinol may be carried out by hydrogenation according to known methods e.g. H₂—Pd/C or NH₄CO₂H—Pd/C. The resulting phenol or pyridinol can then be converted to another group R^{x} as described above.

It will be appreciated by those skilled in the art that it may be necessary to protect certain reactive substituents during some of the above procedures. The skilled person will recognise when a protecting group is required. Standard protection and deprotection techniques, such as those described in Greene T. W. ‘Protective groups in organic synthesis’, New York, Wiley (1981), can be used. For example, carboxylic acid groups can be protected as esters. Deprotection of such groups is achieved using conventional procedures known in the art. It will be appreciated that protecting groups may be interconverted by conventional means.

Cyclohexene intermediates of the formula (VI):

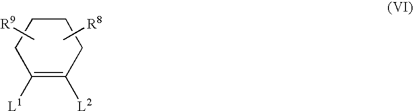

wherein L¹, L² are as defined above, and R⁸ and R⁹ are as hereinbefore defined for compounds of formula (I) are commercially available or may be readily prepared according to known methods.

Compounds of the formula (III):

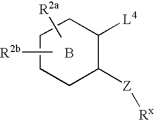

wherein L⁴ is as hereinbefore defined, R^{2a}, R^{2b}, Z, B and R^{x} and are as defined for compounds of formula (I) are commercially available, or may readily be prepared by methods known to those skilled in the art, for example from suitable commercially available pyridinols, anisoles or phenols using methods as described in the examples.

Compounds of the formula (V):

L³-A-R¹P

wherein L³ and P are as defined above and R¹ and A are as hereinbefore defined for compounds of formula (I) are commercially available or may readily be prepared, for example, from suitable halobenzoic acid esters according to known methods, for example using methods as described in the examples.

It is to be understood that the present invention encompasses all isomers of formula (I) and their pharmaceutically acceptable derivatives, including all geometric, tautomeric and optical forms, and mixtures thereof (e.g. racemic mixtures). Where additional chiral centres are present in compounds of formula (I), the present invention includes within its scope all possible diastereoismers, including mixtures thereof. The different isomeric forms may be separated or resolved one from the other by conventional methods, or any given isomer may be obtained by conventional synthetic methods or by stereospecific or asymmetric syntheses.

The compounds of the invention bind to the EP₁ receptor and are therefore considered useful in treating conditions mediated by the action of PGE₂ at EP₁ receptors.

Conditions mediated by the action of PGE₂ at EP₁ receptors include pain; fever; inflammation; immunological diseases; abnormal platelet function diseases; impotence or erectile dysfunction; bone disease; hemodynamic side effects of non-steroidal anti-inflammatory drugs; cardiovascular diseases; neurodegenerative diseases and neurodegeneration; neurodegeneration following trauma; tinnitus; dependence on a dependence-inducing agent; complications of Type I diabetes; and kidney dysfunction.

The compounds of formula (I) are considered to be useful as analgesics. They are therefore considered useful in the treatment or prevention of pain.

The compounds of formula (I) are considered useful as analgesics to treat acute pain, chronic pain, neuropatic pain, inflammatory pain, visceral pain, pain associated with cancer and fibromyalgia, pain associated with migraine, tension headache and cluster headaches, and pain associated with functional bowel disorders, non-cardiac chest pain and non-ulcer dispepsia.

The compounds of formula (I) are considered useful in the treatment of chronic articular pain (e.g. rheumatoid arthritis, osteoarthritis, rheumatoid spondylitis, gouty arthritis and juvenile arthritis) including the property of disease modification and joint structure preservation; musculoskeletal pain; lower back and neck pain; sprains and strains; neuropathic pain; sympathetically maintained pain; myositis; pain associated with cancer and fibromyalgia; pain associated with migraine; pain associated with influenza or other viral infections, such as the common cold; rheumatic fever; pain associated with functional bowel disorders such as non-ulcer dyspepsia, non-cardiac chest pain and irritable bowel syndrome; pain associated with myocardial ischemia; post operative pain; headache; toothache; and dysmenorrhea. The compounds of the invention may also be considered useful in the treatment of visceral pain.

The compounds of the invention are considered to be particularly useful in the treatment of neuropathic pain. Neuropathic pain syndromes can develop following neuronal injury and the resulting pain may persist for months or years, even after the original injury has healed. Neuronal injury may occur in the peripheral nerves, dorsal roots, spinal cord or certain regions in the brain. Neuropathic pain syndromes are traditionally classified according to the disease or event that precipitated them. Neuropathic pain syndromes include: diabetic neuropathy; sciatica; non-specific lower back pain; multiple sclerosis pain; fibromyalgia; HIV-related neuropathy; post-herpetic neuralgia; trigeminal neuralgia; and pain resulting from physical trauma, amputation, cancer, toxins or chronic inflammatory conditions. These conditions are difficult to treat and although several drugs are known to have limited efficacy, complete pain control is rarely achieved. The symptoms of neuropathic pain are heterogeneous and are often described as spontaneous shooting and lancinating pain, or ongoing, burning pain. In addition, there is pain associated with normally non-painful sensations such as “pins and needles” (paraesthesias and dysesthesias), increased sensitivity to touch (hyperesthesia), painful sensation following innocuous simulation (dynamic, static or thermal allodynia), increased sensitivity to noxious stimuli (thermal, cold, mechanical hyperalgesia), continuing pain sensation after removal of the simulation (hyperpathia) or an absence of or deficit in selective sensory pathways (hypoalgesia).

The compounds of formula (I) are also considered useful in the treatment of fever.

The compounds of formula (I) are also considered useful in the treatment of inflammation, for example in the treatment of skin conditions (e.g. sunburn, burns, eczema, dermatitis, psoriasis); ophthalmic diseases such as glaucoma, retinitis, retinopathies, uveitis and of acute injury to the eye tissue (e.g. conjunctivitis); lung disorders (e.g. asthma, bronchitis, emphysema, allergic rhinitis, respiratory distress syndrome, pigeon fanciers disease, farmers lung, chronic obstructive pulmonary disease, (COPD); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation; other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

The compounds of formula (I) are also considered useful in the treatment of immunological diseases such as autoimmune diseases, immunological deficiency diseases or organ transplantation. The compounds of formula (I) are also effective in increasing the latency of HIV infection.

The compounds of formula (I) are also considered useful in the treatment of diseases relating to abnormal platelet function (e.g. occlusive vascular diseases).

The compounds of formula (I) are also considered useful for the preparation of a drug with diuretic action.

The compounds of formula (I) are also considered useful in the treatment of impotence or erectile dysfunction.

The compounds of formula (I) are also considered useful in the treatment of bone disease characterised by abnormal bone metabolism or resorbtion such as osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

The compounds of formula (I) are also considered useful for attenuating the hemodynamic side effects of non-steroidal anti-inflammatory drugs (NSAID's) and cyclooxygenase-2 (COX-2) inhibitors.

The compounds of formula (I) are also considered useful in the treatment of cardiovascular diseases such as hypertension or myocardiac ischemia; functional or organic venous insufficiency, varicose therapy; haemorrhoids; and shock states associated with a marked drop in arterial pressure (e.g. septic shock).

The compounds of formula (I) are also considered useful in the treatment of neurodegenerative diseases and neurodegeneration such as dementia, particularly degenerative dementia (including senile dementia, Alzheimer's disease, Pick's disease, Huntingdon's chorea, Parkinson's disease and Creutzfeldt-Jakob disease, ALS, motor neuron disease); vascular dementia (including multi-infarct dementia); as well as dementia associated with intracranial space occupying lesions; trauma; infections and related conditions (including HIV infection); metabolism; toxins; anoxia and vitamin deficiency; and mild cognitive impairment associated with ageing, particularly Age Associated Memory Impairment.

The compounds of formula (I) are also considered useful in the treatment of neuroprotection and in the treatment of neurodegeneration following trauma such as stroke, cardiac arrest, pulmonary bypass, traumatic brain injury, spinal cord injury or the like.

The compounds of formula (I) are also considered useful in the treatment of tinnitus.

The compounds of formula (I) are also considered useful in preventing or reducing dependence on, or preventing or reducing tolerance or reverse tolerance to, a dependence—inducing agent. Examples of dependence inducing agents include opioids (e.g. morphine), CNS depressants (e.g. ethanol), psychostimulants (e.g. cocaine) and nicotine.

The compounds of formula (I) are also considered useful in the treatment of complications of Type 1 diabetes (e.g. diabetic microangiopathy, diabetic retinopathy, diabetic nephropathy, macular degeneration, glaucoma), nephrotic syndrome, aplastic anaemia, uveitis, Kawasaki disease and sarcoidosis.

The compounds of formula (I) are also considered useful in the treatment of kidney dysfunction (nephritis, particularly mesangial proliferative glomerulonephritis, nephritic syndrome), liver dysfunction (hepatitis, cirrhosis), gastrointestinal dysfunction (diarrhoea) and colon cancer.

The compounds of formula (I) are also useful in the treatment of overactive bladder and urge incontenance.

It is to be understood that reference to treatment includes both treatment of established symptoms and prophylactic treatment, unless explicitly stated otherwise.

According to a further aspect of the invention, we provide a compound of formula (I) or a pharmaceutically acceptable derivative thereof for use in human or veterinary medicine.

According to another aspect of the invention, we provide a compound of formula (I) or a pharmaceutically acceptable derivative thereof for use in the treatment of a condition which is mediated by the action of PGE₂ at EP₁ receptors.

According to a further aspect of the invention, we provide a method of treating a human or animal subject suffering from a condition which is mediated by the action of PGE₂ at EP₁ receptors which comprises administering to said subject an effective amount of a compound of formula (I) or a pharmaceutically acceptable derivative thereof.

According to a further aspect of the invention we provide a method of treating a human or animal subject suffering from a pain, or an inflammatory, immunological, bone, neurodegenerative or renal disorder, which method comprises administering to said subject an effective amount of a compound of formula (I) or a pharmaceutically acceptable derivative thereof.

According to a yet further aspect of the invention we provide a method of treating a human or animal subject suffering from inflammatory pain, neuropathic pain or visceral pain which method comprises administering to said subject an effective amount of a compound of formula (I) or a pharmaceutically acceptable derivative thereof.

According to another aspect of the invention, we provide the use of a compound of formula (I) or a pharmaceutically acceptable derivative thereof for the manufacture of a medicament for the treatment of a condition which is mediated by the action of PGE₂ at EP₁ receptors.

According to another aspect of the invention we provide the use of a compound of formula (I) or a pharmaceutically acceptable derivative thereof for the manufacture of a medicament for the treatment or prevention of a condition such as a pain, or an inflammatory, immunological, bone, neurodegenerative or renal disorder.

According to another aspect of the invention we provide the use of a compound of formula (I) or a pharmaceutically acceptable derivative thereof for the manufacture of a medicament for the treatment or prevention of a condition such as inflammatory pain, neuropathic pain or visceral pain.

The compounds of formula (I) and their pharmaceutically acceptable derivatives are conveniently administered in the form of pharmaceutical compositions. Such compositions may conveniently be presented for use in conventional manner in admixture with one or more physiologically acceptable carriers or excipients.

Thus, in another aspect of the invention, we provide a pharmaceutical composition comprising a compound of formula (I) or a pharmaceutically acceptable derivative thereof adapted for use in human or veterinary medicine.

The compounds of formula (I) and their pharmaceutically acceptable derivatives may be formulated for administration in any suitable manner. They may be formulated for administration by inhalation or for oral, topical, transdermal or parenteral administration. The pharmaceutical composition may be in a form such that it can effect controlled release of the compounds of formula (I) and their pharmaceutically acceptable derivatives.

For oral administration, the pharmaceutical composition may take the form of, for example, tablets (including sub-lingual tablets), capsules, powders, solutions, syrups or suspensions prepared by conventional means with acceptable excipients.

For transdermal administration, the pharmaceutical composition may be given in the form of a transdermal patch, such as a transdermal iontophoretic patch.

For parenteral administration, the pharmaceutical composition may be given as an injection or a continuous infusion (e.g. intravenously, intravascularly or subcutaneously). The compositions may take such forms as suspensions, solutions or emulsions in oily or aqueous vehicles and may contain formulatory agents such as suspending, stabilising and/or dispersing agents. For administration by injection these may take the form of a unit dose presentation or as a multidose presentation preferably with an added preservative. Alternatively for parenteral administration the active ingredient may be in powder form for reconstitution with a suitable vehicle.

The compounds of the invention may also be formulated as a depot preparation. Such long acting formulations may be administered by implantation (for example subcutaneously or intramuscularly) or by intramuscular injection. Thus, for example, the compounds of the invention may be formulated with suitable polymeric or hydrophobic materials (for example as an emulsion in an acceptable oil) or ion exchange resins, or as sparingly soluble derivatives, for example, as a sparingly soluble salt.

The EP₁ receptor compounds for use in the instant invention may be used in combination with other therapeutic agents, for example COX-2 inhibitors, such as celecoxib, deracoxib, rofecoxib, valdecoxib, parecoxib or COX-189; 5-lipoxygenase inhibitors; NSAID's, such as diclofenac, indomethacin, nabumetone or ibuprofen; leukotriene receptor antagonists; DMARD's such as methotrexate; adenosine A1 receptor agonists; sodium channel blockers, such as lamotrigine; NMDA receptor modulators, such as glycine receptor antagonists; gabapentin and related compounds; tricyclic antidepressants such as amitriptyline; neurone stabilising antiepileptic drugs; mono-aminergic uptake inhibitors such as venlafaxine; opioid analgesics; local anaesthetics; 5HT₁ agonists, such as triptans, for example sumatriptan, naratriptan, zolmitriptan, eletriptan, frovatriptan, almotriptan or rizatriptan; nicotinic acetyl choline (nACh) receptor modulators; glutamate receptor modulators, for example modulators of the NR2B subtype; EP₄ receptor ligands; EP₂ receptor ligands; EP₃ receptor ligands; EP₄ antagonists; EP₂ antagonists and EP₃ antagonists; cannabanoid receptor ligands; bradykinin receptor ligands and vanilloid receptor ligand. When the compounds are used in combination with other therapeutic agents, the compounds may be administered either sequentially or simultaneously by any convenient route.

Additional COX-2 inhibitors are disclosed in U.S. Pat. No. 5,474,995 U.S. Pat. No. 5,633,272; U.S. Pat. No. 5,466,823, U.S. Pat. No. 6,310,099 and U.S. Pat. No. 6,291,523; and in WO 96/25405, WO 97/38986, WO 98/03484, WO 97/14691, WO99/12930, WO00/26216, WO00/52008, WO00/38311, WO01/58881 and WO02/18374.

The invention thus provides, in a further aspect, a combination comprising a compound of formula (I) or a pharmaceutically acceptable derivative thereof together with a further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable derivative thereof is used in combination with a second therapeutic agent active against the same disease state the dose of each compound may differ from that when the compound is used alone. Appropriate doses will be readily appreciated by those skilled in the art.

A proposed daily dosage of compounds of formula (I) or their pharmaceutically acceptable derivatives for the treatment of man is from 0.01 to 30 mg/kg body weight per day and more particularly 0.1 to 10 mg/kg body weight per day, which may be administered as a single or divided dose, for example one to four times per day. The dose range for adult human beings is generally from 8 to 2000 mg/day, such as from 20 to 1000 mg/day, preferably 35 to 200 mg/day.

The precise amount of the compounds of formula (I) administered to a host, particularly a human patient, will be the responsibility of the attendant physician. However, the dose employed will depend on a number of factors including the age and sex of the patient, the precise condition being treated and its severity, and the route of administration.

No unacceptable toxicological effects are expected with compounds of the invention when administered in accordance with the invention.

All publications, including but not limited to patents and patent applications, cited in this specification are herein incorporated by reference as if each individual publication were specifically and individually indicated to be incorporated by reference herein as though fully set forth.

The following non-limiting Examples illustrate the preparation of pharmacologically active compounds of the invention.

### EXAMPLES

### Abbreviations

Bn (benzyl), Bu, Pr, Me, Et (butyl, propyl, methyl ethyl), DMSO (dimethyl sulfoxide), DCM (dichloromethane), DMF (N,N-dimethylformamide), EDTA (ethylenediaminetetraacetic acid), EtOAc (ethyl acetate), EtOH (ethanol), HPLC (High pressure liquid chromatography), LCMS (Liquid chromatography/Mass spectroscopy), MDAP (Mass Directed Purification), MeCN (acetonitrile), MeOH (methanol), NMR (Nuclear Magnetic Resonance (spectrum)), Ph (phenyl), pTSA (para-toluene sulphonic acid), SPE (Solid Phase Extraction), TBAF (tetrabutylammonium fluoride), THF (tetrahydrofuran), s, d, t, q, m, br (singlet, doublet, triplet, quartet, multiplet, broad.)

### LCMS

Column: 3.3 cm×4.6 mm ID, 3 um ABZ+PLUS
Flow Rate: 3 ml/min
Injection Volume: 5 μl
Temp: RT
UV Detection Range: 215 to 330 nm
   Solvents: A: 0.1% Formic Acid+10 mMolar Ammonium Acetate.
B: 95% Acetonitrile+0.05% Formic Acid

Gradient:

| Time | A % | B % |
|---|---|---|
| 0.00 | 100 | 0 |
| 0.70 | 100 | 0 |
| 4.20 | 0 | 100 |
| 5.30 | 0 | 100 |
| 5.50 | 100 | 0 |

### Mass Directed Autopreparation

### Hardware:

Waters 600 gradient pump
Waters 2767 inject/collector

### Waters Reagent Manager

Micromass ZMD mass spectrometer
Gilson Aspec—waste collector
Gilson 115 post-fraction UV detector

### Software:

Micromass Masslynx version 4.0

### Column

The column used is typically a Supelco LCABZ++ column whose dimensions are 20 mm internal diameter by 100 mm in length. The stationary phase particle size is 5 μm.

### Solvents:

A: Aqueous solvent=Water+0.1% Formic Acid
B: Organic solvent=MeCN:Water 95:5+0.05% Formic Acid
Make up solvent=MeOH:Water 80:20+50 mMol Ammonium Acetate
Needle rinse solvent=MeOH:Water DMSO 80:10:10

The method used depends on the analytical retention time of the compound of interest. 15-minute runtime, which comprises a 10-minute gradient followed by a 5-minute column flush and re-equilibration step.

### MDP 1.5-2.2=0-30% B

### MDP 2.0-2.8=5-30% B

### MDP 2.5-3.0=15-55% B

### MDP 2.8-4.0=30-80% B

### MDP 3.8-5.5=50-90% B

### Flow Rate:

flow rate 20 ml/min.

### Intermediates

### 2-Benzyloxy-5-chloroiodobenzene

A mixture of 5-chloro-2-iodophenol (20 g, 78.59 mmol), potassium carbonate (20 g, 144.93 mmol) and benzyl bromide (14.78 g, 86.45 mmol) in acetone (200 ml) was stirred and refluxed for 3 hours. The resulting mixture was cooled, filtered, evaporated to dryness, dissolved in iso-hexane and left to crystallise. There was obtained 25.74 g of light brown solid.

¹H NMR (CDCl₃) δ: 5.13 (s, 2H), 6.76 (d, 1H), 7.22-7.48 (m, 6H), 7.77 (d, 1H).

### 2-Benzyloxy-5-chlorophenylboronic acid

A solution of 2M isopropylmagnesium chloride in diethyl ether (46.5 ml, 93 mmol) was added over 20 minutes to a solution of 2-benzyloxy-5-chloroiodobenzene (16.02 g, 46.5 mmol) in dry THF (150 ml) at −40° C. under nitrogen. After stirring for 1 hour at −40° C. the mixture was cooled to −78° C. and trimethyl borate (9.67 g, 93 mmol) was added over 5 minutes. The resulting mixture was allowed to warm to room temperature and 2M hydrochloric acid (200 ml) was added and stirred vigorously for 15 minutes. The organic phase was separated, dried (magnesium sulphate), evaporated to dryness and triturated with iso-hexane to give 11.9 g of off-white solid. LC/MS: Rt 3.4, [2MH−] 637.3.

### 2-(2-Bromo-1-cyclohexen-1-yl)-4-chloro-1-[(Phenylmethyl)oxy]benzene

A mixture of 2-benzyloxy-5-chlorophenylboronic acid (2.983 g, 11.36 mmol), 1,2-dibromocyclohexene (10.91 g, 45.46 mmol), potassium carbonate (13.8 g, 100 mmol) and tetrakis(triphenylphosphine)palladium(0) (1.158 g, 1 mmol) in 1:1 toluene/ethanol (250 ml) was stirred and heated at 90° C. under nitrogen for 4 hours. After cooling the mixture was diluted with diethyl ether and water and the organic layer separated, dried (magnesium sulphate) and evaporated to dryness. The residue was chromatographed on silica eluting with ethyl acetate/iso-hexane (1:49) then rechromatographed eluting with dichloromethane/iso-hexane (1:9) and recrystallised from iso-hexane to give 638 mg of white crystals.

¹H NMR (d₆ DMSO) δ: 1.55-1.80 (m, 4H), 2.12-2.22 (m, 1H), 2.30-2.41 (m, 1H), 2.51-2.60 (m, 2H), 5.13 (s, 2H), 7.07 (d, 1H), 7.10 (d, 1H), 7.28-7.43 (m, 6H).

### (2-{5-Chloro-2-[(phenylmethyl)oxy]phenyl}-1-cyclohexan-1-yl) boronic acid

1.6M butyllithium in hexanes (0.21 ml, 0.34 mmol) was added to a stirred solution of 2-(2-bromo-1-cyclohexen-1-yl)-4-chloro-1-[(phenylmethyl)oxy]benzene (126 mg, 0.33 mmol) in dry THF (5 ml) at −78° C. under nitrogen and stirred for 15 minutes. Triisopropyl borate (124 mg, 0.66 mmol) was added and the mixture allowed to warm to room temperature and 2M hydrochloric acid (5 ml) was added. The resulting mixture was stirred vigorously for 10 minutes and the organic layer separated, dried (magnesium sulphate) evaporated and chromatographed on silica eluting with ethyl acetate/iso-hexane (1:4) to give 44 mg of colourless gum. LC/MS: Rt 3.73, [2MH+] 689.4.

### Ethyl 6-(2-{5-chloro-2-[(phenylmethyl)oxy]phenyl}-1-cyclohexen-1-yl)-2-pyridinecarboxylate

A mixture of (2-{5-chloro-2-[(phenylmethyl)oxy]phenyl}-1-cyclohexen-1-yl)boronic acid (44 mg, 0.128 mmol), 6-bromopicolinic acid ethyl ester (30 mg, 0.13 mmol), potassium carbonate (138 mg, 1 mmol) and tetrakis(triphenylphosphine)palladium(0) (12 mg, 0.01 mmol) in 1:1 toluene/ethanol (3 ml) was stirred and heated at 90° C. under nitrogen for 2 hours. After cooling the mixture was diluted with diethyl ether and water and the organic layer separated, dried (magnesium sulphate) and evaporated to dryness then chromatographed on silica eluting with ethyl acetate/iso-hexane (1:9) to give 35 mg of colourless gum. LC/MS: Rt 4.16, [MH+] 448.4, 450.4.

### Ethyl 6-[2-(5-chloro-2-hydroxyphenyl)-1-cyclohexen-1-yl]-2-pyridinecarboxylate

Ethyl 6-(2-{5-chloro-2-[(phenylmethyl)oxy]phenyl}-1-cyclohexen-1-yl)-2-pyridinecarboxylate (310 mg, 0.69 mmol) was dissolved in acetic acid (1 ml) and 48% hydrogen bromide in acetic acid (5 ml) and left at room temperature for 1 hour then poured into diethyl ether/water and basified with potassium carbonate. The organic layer was separated, dried (magnesium sulphate) and chromatographed on silica eluting with ethyl acetate/iso-hexane (3:17) and the resulting mixture dissolved in ethanol (5 ml) and 60% sodium hydride (2 mg) added. After leaving overnight at room temperature the solution was diluted with water/diethyl ether, acidified with acetic acid and the organic layer washed with saturated sodium bicarbonate solution, dried (magnesium sulphate) and evaporated to give 201 mg of colourless gum. LC/MS: Rt 3.56, [MH+] 358.4, 360.4.

### Ethyl 6-[2-(5-chloro-2-{[(4-fluorophenyl)methyl]oxy}phenyl)-1-cyclohexen-1-yl]-2-pyridinecarboxylate

A mixture of ethyl 6-[2-(5-chloro-2-hydroxyphenyl)-1-cyclohexen-1-yl]-2-pyridinecarboxylate (100 mg, 0.28 mmol), 4-fluorobenzyl bromide (63 mg, 0.33 mmol) and potassium carbonate (138 mg, 1 mmol) in acetone (5 ml) was stirred and refluxed for 3 hours. After cooling the mixture was filtered, evaporated and chromatographed on silica eluting with ethyl acetate/iso-hexane (1:9) to give 96 mg of white solid. LC/MS: Rt 4.17, [MH+] 466.4, 468.3.

### Ethyl 6-[2-(5-chloro-2-{[(2,4-difluorophenyl)methyl]oxy}phenyl)-1-cyclohexen-1-yl]-2-pyridinecarboxylate

Prepared as a white solid in the same manner as for ethyl 6-[2-(5-chloro-2-{[(4-fluorophenyl)methyl]oxy}phenyl)-1-cyclohexen-1-yl]-2-pyridinecarboxylate but using 2,4-difluorobenzyl bromide instead of 4-fluorobenzyl bromide. LC/MS: Rt 4.22, [MH+] 484.4, 486.3.

### Example 1

### 6-[2-(5-Chloro-2-{[(2,4-difluorophenyl)methyl]oxy}phenyl)-1-cyclohexen-1-yl]-2-pyridinecarboxylic acid

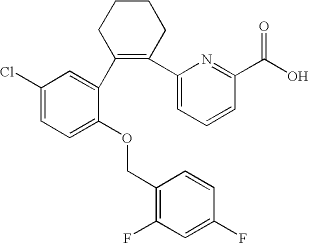

Ethyl 6-[2-(5-chloro-2-{[(2,4-difluorophenyl)methyl]oxy}phenyl)-1-cyclohexen-1-yl]-2-pyridinecarboxylate (116 mg, 0.24 mmol) was dissolved in ethanol (5 ml) and 2M sodium hydroxide (1 ml) and left at room temperature for 1 hour. The resulting solution was diluted with water/diethyl ether, acidified with acetic acid and the organic layer separated, dried (magnesium sulphate), toluene (10 ml) added and evaporated to dryness. The residue was triturated with iso-hexane to give 79 mg of white solid. LC/MS: Rt 3.91, [MH+] 456.3, 458.3.

¹H NMR (d₆ DMSO) δ: 1.60-1.82 (4H, br.m.), 2.05-2.85 (4H, br. m.), 5.11 (2H, s), 6.76 (1H, d), 6.88 (1H, dd), 7.08-7.19 (3H, m), 7.27-7.34 (1H, dt), 7.52-7.59 (2H, m), 7.25 (1H, d), 12.7-13.4 (1H, br. s.).

### Example 2

### 6-[2-(5-Chloro-2-{[(4-fluorophenyl)methyl]oxy}phenyl)-1-cyclohexen-1-yl]-2-pyridinecarboxylic acid

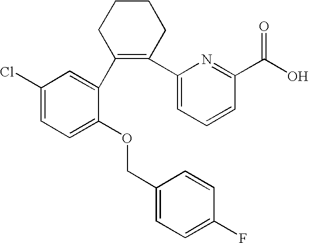

Ethyl 6-[2-(5-chloro-2-{[(4-fluorophenyl)methyl]oxy}phenyl)-1-cyclohexen-1-yl]-2-pyridinecarboxylate (96 mg, 0.21 mmol) was dissolved in ethanol (5 ml) and 2M sodium hydroxide (1 ml) and left at room temperature for 1 hour. The resulting solution was diluted with water/diethyl ether, acidified with acetic acid and the organic layer separated, dried (magnesium sulphate), toluene (10 ml) added and evaporated to dryness. The residue was triturated with iso-hexane to give 72 mg of white solid.

LC/MS: Rt 3.80, [MH+] 438.3, 440.4

¹H NMR (d₆ DMSO) δ: 1.69-1.83 (4H, m), 2.1-2.8 (4H, br.m), 5.07 (2H, s), 6.79 (1H, d), 6.91 (1H, dd), 7.00 (1H, d), 7.16 (1H, dd), 7.20 (2H, t), 7.43 (2H, dd), 7.55 (1H, t), 7.72 (1H, d), 12.5-13.4 (1H, br.s.).

### Example 3

### 6-(2-{5-Chloro-2-[(phenylmethyl)oxy]phenyl}-1-cyclohexen-1-yl)-2-pyridinecarboxylic acid sodium salt

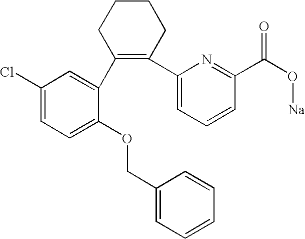

Ethyl 6-(2-{5-chloro-2-[(phenylmethyl)oxy]phenyl}-1-cyclohexen-1-yl)-2-pyridinecarboxylate (60 mg, 0.13 mmol) was dissolved in ethanol (5 ml) and 2M sodium hydroxide (1 ml) and left at room temperature for 1 hour. The resulting solution was diluted with water/diethyl ether, acidified with acetic acid and the organic layer separated, dried (magnesium sulphate), toluene (10 ml) added and evaporated to dryness. The residue was dissolved in ethanol (2 ml) and 2M sodium hydroxide (0.5 ml) then diluted with ethyl acetate/water. The organic layer was dried and evaporated to give 11 mg of white solid.

LC/MS: Rt 3.83, [MH+] 420.4, 422.3

¹H NMR (de DMSO) (50° C.) δ: 1.66-1.79 (4H, m), 2.19-2.25 (2H, m), 2.46-2.54 (2H, m), 5.09 (2H, s), 6.75 (1H, d), 6.78 (1H, d), 6.93 (1H, d), 6.99 (1H, dd), 7.26-7.46 (6H, m), 7.75 (1H, d).

### Intermediates

### 4-(Benzyloxy)benzotrifluoride

A solution of 4-hydroxybenzotrifluoride (8.55 g, 52.78 mmol) in acetone (200 ml) was treated with benzyl bromide (9.87 g, 6.86 ml, 58.05 mmol) and potassium carbonate (10.94 g, 79.16 mmol). The mixture was stirred and heated to reflux under nitrogen for 3 h. After cooling, diethyl ether (400 ml) and water (400 ml) were added and the aqueous phase re-extracted with diethyl ether (100 ml). The combined organic layers were washed with water, dried (MgSO₄) and the solvent removed in vacuo to leave the title compound as a white solid. (12.71 g, 95%)

¹H NMR (CDCl₃) δ: 5.11 (2H, s), 7.03 (2H, d, J=9 Hz), 7.34-7.44 (5H, m), 7.55 (2H, d, J=9 Hz).

### 2-Benzyloxy-5-(trifluoromethyl)iodobenzene

A solution of 4-(benzyloxy)benzotrifluoride (12.71 g, 50.4 mmol) in acetonitrile (300 ml) was stirred under nitrogen and 1-(chloromethyl)-4-fluoro-1,4-diazoniabicyclo[2.2.2]octane bis(tetrafluoroborate) (17.75 g, 50.4 mmol) and iodine (6.4 g, 25.2 mmol) added. The mixture was stirred at room temperature for 88 h. The solvent was evaporated and the residue partitioned between ethyl acetate (400 ml) and water (400 ml). The organic layer was washed with water, dried (MgSO₄) and evaporated to an orange oil which was purified by flash chromatography (silica gel, 5% ethyl acetate:isohexane) to give the title compound as an orange oil (15.07 g, 79%)

¹H NMR (CDCl₃) δ: 5.21 (2H, s), 6.89 (1H, d J=9 Hz), 7.32-7.55 (6H, m), 8.04 (1H, d, J=2 Hz).

### 1-[2-[(Phenylmethyl)oxy]-5-(trifluoromethyl)phenyl]cyclohexanol

A solution of cyclohexanone (490 mg, 5 mmol) in anhydrous diethyl ether (5 ml) was added with ice bath cooling to a solution of the Grignard prepared from magnesium (122 mg, 5 mmol) and 2-benzyloxy-5-(trifluoromethyl)iodobenzene (1.895 g, 5 mmol) in anhydrous diethyl ether (10 ml) and the mixture stirred for 30 mins before being quenched with saturated ammonium chloride. The organic layer was dried (magnesium sulphate) evaporated and the residue purified by flash chromatography on silica gel eluting with ethyl acetate/iso-hexane (7:93) to give the title compound as a white solid (1.04 g).

LC/MS: Rt=3.6 min

### 2-(1-Cyclohexen-1-yl)-1-[(phenylmethyl)oxy]-4-(trifluoromethyl)benzene

A mixture of 1-[2-[(phenylmethyl)oxy]-5-(trifluoromethyl)phenyl]cyclohexanol (1 g, 2.86 mmol) and 4-toluenesulphonic acid (10 mg, 0.05 mmol) in toluene (15 ml) was stirred and refluxed for 1 hour then cooled, washed with saturated sodium bicarbonate solution, dried (magnesium sulphate) and evaporated to give the title compound as a light brown oil (951 mg). LC/MS: Rt=3.7 min.

### 1-[2-[(Phenylmethyl)oxy]-5-(trifluoromethyl)phenyl]-7-oxabicyclo[4.1.0]heptane

A solution of 2-(1 cyclohexen-1-yl)-1-[(phenylmethyl)oxy]-4-(trifluoromethyl)benzene (950 mg, 2.86 mmol) and 3-chloroperbenzoic acid (641 mg, 77%, 2.86 mmol) was left at room temperature for 3 hours then washed with saturated sodium bisulphite and saturated sodium bicarbonate solution before being dried (magnesium sulphate) and evaporated. The residue was purified by flash chromatography on silica gel eluting with ethyl acetate/iso-hexane (5:95) to give the title compound as a colourless oil (890 mg).

LC/MS: Rt=3.85 min.

### 2-[2-[(Phenylmethyl)oxy]-5-(trifluoromethyl)phenyl]cyclohexanone

A mixture of 1-[2-[(phenylmethyl)oxy]-5-(trifluoromethyl)phenyl]-7-oxabicyclo[4.1.0]heptane (21.7 g. 62.36 mmol) and indium (III) chloride (13.78 g, 62.36 mmol) in THF (200 ml) was stirred and heated at 60° C. under nitrogen for 18 hours then diluted with diethyl ether and water. The organic phase was dried (magnesium sulphate) and evaporated and the residue triturated with cold iso-hexane and the white solid filtered off to give to give the title compound (9.5 g). LC/MS: Rt=3.5 min.

### 2-[2-[(Phenylmethyl)oxy]-5-(trifluoromethyl)phenyl]-1-cyclohexen-1-yl trifluoromethanesulfonate

A solution of 2-[2-[(phenylmethyl)oxy]-5-(trifluoromethyl)phenyl]cyclohexanone (2.088 g, 6 mmol) in dry dimethylformamide (5 ml) was added dropwise to a stirred suspension of 60% sodium hydride (460 mg, 11.5 mmol) in dimethylformamide (12.5 ml) with water bath cooling and the resulting mixture stirred at room temperature for 2 hours. N-phenyltrifluoromethanesulphonimide (2.49 g, 6.9 mmol) was added in one portion and stirred overnight before being carefully diluted with diethyl ether/water and the organic phase was washed with water (×3) dried (magnesium sulphate) and evaporated. The residue was purified by chromatography on silica gel eluting with ethyl acetate/iso-hexane (1:49) to give the title compound as a colourless oil (1.67 g). LC/MS: Rt=3.9 min.

### 4,4,5,5-Tetramethyl-2{-2-[2-[(phenylmethyl)oxy]-5-(trifluoromethyl)phenyl]-1-cyclohexen-1-yl}-1,3,2-dioxaborolane

A mixture of 2-[2-[(phenylmethyl)oxy]-5-(trifluoromethyl)phenyl]-1-cyclohexen-1-yl trifluoromethanesulfonate (1.65 g, 3.43 mmol), bis(pinacolato)diboron (960 mg, 3.78 mmol), potassium carbonate (712 mg, 5.16 mmol), triphenylphosphine (54 mg, 0.21 mmol) and bis(triphenylphosphine)palladium(II) chloride (73 mg, 0.105 mmol) in dioxan (20 ml) was stirred and heated at 80° C. under nitrogen for 18 hours then cooled and diluted with diethyl ether/water. The organic phase was dried (magnesium sulphate), evaporated. and the residue purified by chromatography on silica gel eluting with ethyl acetate/iso-hexane (1:49) to give the title compound as a colourless oil (1.38 g). LC/MS: Rt=4.1 min.

### {2-[2-[(Phenylmethyl)oxy]-5-(trifluoromethyl)phenyl]-1-cyclohexen-1-yl}boronic acid

4,4,5,5-Tetramethyl-2-{2-[2-[(phenylmethyl)oxy]-5-(trifluoromethyl)phenyl]-1-cyclohexen-1-yl}-1,3,2-dioxaborolane (4.2 g, 9.16 mmol) was dissolved in a mixture of ethanol (40 ml) and 2M sodium hydroxide (20 ml) and left at room temperature for 30 hours. The ethanol was evaporated off and the residue acidified with 2M hydrochloric acid and extracted with diethyl ether which was dried (magnesium sulphate) and evaporated to give the title compound as a colourless oil (3.4 g). LC/MS: Rt=3.29 min.

### Ethyl 6-{2-[2-[(phenylmethyl)oxy]-5-(trifluoromethyl)phenyl]-1-cyclohexen-1-yl}-2-pyridinecarboxylate

A mixture of {2-[2-[(phenylmethyl)oxy]-5-(trifluoromethyl)phenyl]-1-cyclohexen-1-yl}boronic acid (1.504 g, 4 mmol), ethyl 6-bromopicolinate (1.15 g, 5 mmol), potassium carbonate (2.76 g, 20 mmol) and tetrakis(triphenylphosphine)palladium(0) (463 mg, 0.4 mmol) in 1:1 toluene/ethanol (30 ml) was stirred and heated at 90° C. under nitrogen for 3 hours then cooled, diluted with water/diethyl ether and the organic phase dried (magnesium sulphate) and evaporated. The residue was purified by chromatography on silica gel eluting with ethyl acetate/iso-hexane (8:92) to give the title compound as a white solid. (1.089).

LC/MS: Rt=4.02 min.

### Ethyl 5-{2-[2-[(Phenylmethyl)oxy]-5-(trifluoromethyl)phenyl]-1-cyclohexen-1-yl}-3-pyridinecarboxylate

Prepared as an off-white solid by the same method as for ethyl 6-{2-[2-[(phenylmethyl)oxy]-5-(trifluoromethyl)phenyl]-1-cyclohexen-1-yl}-2-pyridinecarboxylate but using ethyl 5-bromonicotinate instead of ethyl 6-bromopicolinate. LC/MS: Rt=3.63 min.

### Ethyl 6-{2-[2-hydroxy-5-(trifluoromethyl)phenyl]-1-cyclohexen-1-yl}-2-pyridinecarboxylate

Ethyl 6-{2-[2-[(phenylmethyl)oxy]-5-(trifluoromethyl)phenyl]-1-cyclohexen-1-yl}-2-pyridinecarboxylate (1.07 g, 2.22 mmol) was dissolved in 48% hydrogen bromide in acetic acid (10 ml) and left at room temperature for 3 hours then diluted with diethyl ether/water, basified with potassium carbonate and chromatographed on silica gel with ethyl acetate/iso-hexane (1:4). The resulting mixture was dissolved in ethanol (20 ml) and 60% sodium hydride (5 mg) added. After standing overnight the solution was evaporated, dissolved in diethyl ether/water and acidified with acetic acid. The organic phase was washed with saturated sodium bicarbonate, dried (magnesium sulphate), evaporated and the residue triturated with iso-hexane to give the title compound as a white solid (740 mg). LC/MS: Rt=3.37 min.

### Ethyl 5-{2-[2-hydroxy-5-(trifluoromethyl)phenyl]-1-cyclohexen-1-yl}-3-pyridinecarboxylate

Prepared by the same method as for ethyl 6-{2-[2-hydroxy-5-(trifluoromethyl)phenyl]-1-cyclohexen-1-yl}-2-pyridinecarboxylate but using ethyl 5-{2-[2-[(phenylmethyl)oxy]-5-(trifluoromethyl)phenyl]-1-cyclohexen-1-yl}-3-pyridinecarboxylate instead of ethyl 6-{2-[2-[(phenylmethyl)oxy]-5-(trifluoromethyl)phenyl]-1-cyclohexen-1-yl}-2-pyridinecarboxylate. LC/MS: Rt=3.57 min.

### 4-Chloro-2-(1-cyclohexen-1-yl)-1-(methyloxy)benzene

A solution of cyclohexanone (4.9 g, 50 mmol) in anhydrous ether (20 ml) was added to the Grignard prepared from 4-chloro-2-iodoanisole (13.43 g, 50 mmol) and magnesium (1.215 g, 5 mmol) in anhydrous ether (100 ml) with ice cooling and stirred for one hour. After addition of saturated ammonium chloride the organic phase was dried (magnesium sulphate) evaporated and triturated with iso-hexane to give a white solid (10.1 g) which was dissolved in toluene (100 ml) and toluenesulphonic acid monohydrate (50 mg) added. After refluxing for 2 hours the solution was cooled, washed with saturated sodium bicarbonate, dried (magnesium sulphate) and evaporated to give the title compound as a yellow oil (8.7 g). LC/MS: Rt=4.15 min.

### 1-[5-chloro-2-(methyloxy)phenyl]-7-oxabicyclo[4.1.0]heptane

Prepared by the same method as for 1-[2-[(phenylmethyl)oxy]-5-(trifluoromethyl)phenyl]-7-oxabicyclo[4.1.0]heptane but using 4-chloro-2-(1-cyclohexen-1-yl)-1-(methyloxy)benzene instead of 2-(1-cyclohexen-1-yl)-1-[(phenylmethyl)oxy]-4-(trifluoromethyl)benzene. LC/MS: Rt=3.20 min.

### 2-[5-Chloro-2-(methyloxy)phenyl]cyclohexanone

Prepared by the same method as for 2-[2-[(phenylmethyl)oxy]-5-(trifluoromethyl)phenyl]cyclohexanone but using 1-[5-chloro-2-(methyloxy)phenyl]-7-oxabicyclo[4.1.0]heptane instead of 1-[2-[(phenylmethyl)oxy]-5-(trifluoromethyl)phenyl]-7-oxabicyclo[4.1.0]heptane. LC/MS: Rt=3.03 min.

### 2-[5-Chloro-2-(methyloxy)phenyl]-1-cyclohexen-1-yl trifluoromethanesulfonate

Prepared by the same method as for 2-[2-[(phenylmethyl)oxy]-5-(trifluoromethyl)phenyl]-1-cyclohexen-1-yl trifluoromethanesulfonate but using 2-[5-chloro-2-(methyloxy)phenyl]cyclohexanone instead of 2-[2-[(phenylmethyl)oxy]-5-(trifluoromethyl)phenyl]cyclohexanone. LC/MS: Rt=4.08 min.

### 2-{2-[5-Chloro-2-(methyloxy)phenyl]-1-cyclohexen-1-yl}-4,4,5,5-tetramethyl-1,3,2-dioxaborolane

Prepared by the same method as for 4,4,5,5-tetramethyl-2-{2-[2-[(phenylmethyl)oxy]-5-(trifluoromethyl)phenyl]-1-cyclohexen-1-yl}-1,3,2-dioxaborolane but using 2-[5-chloro-2-(methyloxy)phenyl]-1 cyclohexen-1-yl trifluoromethanesulfonate instead of 2-[2-[(phenylmethyl)oxy]-5-trifluoromethyl)phenyl]-1-cyclohexen-1-yl trifluoromethanesulfonate. LC/MS: Rt=4.30 min.

### {2-[5-Chloro-2-(methyloxy)phenyl]-1-cyclohexen-1-yl}boronic acid

Prepared by the same method as for {2-[2-[(phenylmethyl)oxy]-5-(trifluoromethyl)phenyl]-1-cyclohexen-1-yl}boronic acid but using 2-{2-[5-chloro-2-(methyloxy)phenyl]-1-cyclohexen-1-yl}-4,4,5,5-tetramethyl-1,3,2-dioxaborolane instead of 4,4,5,5-tetramethyl-2-{2-[2-[(phenylmethyl)oxy]-5-(trifluoromethyl)phenyl]-1-cyclohexen-1-yl}-1,3,2-dioxaborolane. LC/MS: Rt=3.13 min.

### Ethyl 6-{2-[5-chloro-2-(methyloxy)phenyl]-1-cyclohexen-1-yl}-2-pyridinecarboxylate

Prepared by the same method as for ethyl 6-{2-[2-[(phenylmethyl)oxy]-5-(trifluoromethyl)phenyl]-1-cyclohexen-1-yl}-2-pyridinecarboxylate but using {2-[5-chloro-2-(methyloxy)phenyl]-1-cyclohexen-1-yl}boronic acid instead of {2-[2-[(phenylmethyl)oxy]-5-(trifluoromethyl)phenyl]-1-cyclohexen-1-yl}boronic acid. LC/MS: Rt=4.05 min.

### Ethyl 5-{2-[5-chloro-2-(methyloxy)phenyl]-1-cyclohexen-1-yl}-3-pyridinecarboxylate

Prepared by the same method as for ethyl 6-{2-[2-[(phenylmethyl)oxy]-5-(trifluoromethyl)phenyl]-1-cyclohexen-1-yl}-2-pyridinecarboxylate but using 2-[5-chloro-2-(methyloxy)phenyl]-1-cyclohexen-1-yl}boronic acid instead of {2-[2-[(phenylmethyl)oxy]-5-(trifluoromethyl)phenyl]-1-cyclohexen-1-yl}boronic acid and 5-bromonicotinic acid ethyl ester instead of 6-bromopicolinic acid ethyl ester. LC/MS: Rt=3.70 min.

### Ethyl 3-{2-[5-chloro-2-(methyloxy)phenyl]-1-cyclohexen-1-yl}2-pyridinecarboxylate

Prepared by the same method as for ethyl 6-{2-[2-[(phenylmethyl)oxy]-5-(trifluoromethyl)phenyl]-1-cyclohexen-1-yl-2-pyridinecarboxylate but using 2-[5-chloro-2-(methyloxy)phenyl]-1-cyclohexen-1-yl}boronic acid instead of {2-[2-[(phenylmethyl)oxy]-5-(trifluoromethyl)phenyl]-1-cyclohexen-1-yl}boronic acid and 3-iodonicotinic acid methyl ester instead of 6-bromopicolinic acid ethyl ester. LC/MS: Rt=3.73 min.

### Ethyl 6-[2-(5-chloro-2-hydroxyphenyl)-1-cyclohexen-1-yl]-2-pyridinecarboxylate

Ethyl 6-{2-[5-chloro-2-(methyloxy)phenyl]-1-cyclohexen-1-yl}-2-pyridinecarboxylate (900 mg, 2.42 mmol) was dissolved in 1M boron tribromide in dichloromethane (10 ml) and left at room temperature for 1 hour then poured onto ice/ether, basified with 2M sodium hydroxide solution and acidified with acetic acid. The organic phase was dried, evaporated, re-evaporated with toluene then dissolved in ethanol (25 ml) and sulphuric acid (2 ml) and refluxed for 20 hours. The resulting solution was cooled evaporated, dissolved in water/ether and basified with potassium carbonate. The organic phase was dried, evaporated and the residue was chromatographed on silica gel eluting with ethyl acetate/iso-hexane (3:17) to give the title compound as a white solid (705 mg).

LC/MS: Rt=3.44 min.

### Ethyl 5-[2-(5-chloro-2-hydroxyphenyl)-1-cyclohexen-1-yl]-3-pyridinecarboxylate

Prepared by the same method as for ethyl 6-[2-(5-chloro-2-hydroxyphenyl)-1-cyclohexen-1-yl]-2-pyridinecarboxylate but using ethyl 5-{2-[5-chloro-2-(methyloxy)phenyl]-1-cyclohexen-1-yl}3-pyridinecarboxylate instead of ethyl 6-{2-[5-chloro-2-(methyloxy)phenyl]-1-cyclohexen-1-yl}2-pyridinecarboxylate. LC/MS: Rt=3.38 min.

### Ethyl 3-[2-(5-chloro-2-hydroxyphenyl)-1-cyclohexen-1-yl]-2-pyridinecarboxylate

Prepared by the same method as for ethyl 6-[2-(5-chloro-2-hydroxyphenyl)-1-cyclohexen-1-yl]-2-pyridinecarboxylate but using ethyl 3-{2-[chloro-2-(methyloxy)phenyl]-1-cyclohexen-1-yl}-2-pyridinecarboxylate instead of ethyl 6-{2-[5-chloro-2-(methyloxy)phenyl]-1-cyclohexen-1-yl}-2-pyridinecarboxylate. LC/MS: Rt=3.52 min.

### Ethyl 4-chloro-2-pyridinecarboxylate 1-oxide

A solution of ethyl 4-chloro-2-pyridinecarboxylate (15.6 g, 84.1 mmol) and 77% 3-chloroperbenzoic acid (100 mmol) in dichloromethane (150 ml) was left at room temperature for 2 days then washed with saturated sodium bisulpite solution and saturated sodium bicarbonate solution. The aqueous washes were extracted (×3) with dichloromethane and the combined organic extracts dried (magnesium sulphate) evaporated and the residue triturated with iso-hexane to give the title compound as a white solid (13.95 g). LC/MS: Rt=1.48 min.

### Ethyl 4-chloro-6-(2-hydroxy-1-cyclohexen-1-yl)-2-pyridinecarboxylate

Benzoyl chloride (9.71 g, 69.11 mmol) was added to a solution of ethyl 4-chloro-2-pyridinecarboxylate 1-oxide (13.93 g, 69.11 mmol) in dichloromethane (175 ml) with ice cooling and stirred for 30 minutes. 1-(4-morpholino)cyclohexene (13.84 g, 82.87 mmol) was added and the mixture refluxed for 1 hour, cooled, washed with saturated sodium bicarbonate and the organic evaporated. The residue was dissolved in ether/2M hydrochloric acid and yellow solid separated. After diluting with dichloromethane and basifying with potassium carbonate the organic phase was dried (magnesium sulphate), evaporated and the residue chromatographed on silica gel eluting with ethyl acetate/iso-hexane (1:9) to give the title compound as a yellow solid (7.52 g). LC/MS: Rt=3.97 min.

### Ethyl 6-(2-hydroxy-1-cyclohexen-1-yl)-2-pyridinecarboxylate

A solution of ethyl 4-chloro-6-(2-hydroxy-1-cyclohexen-1-yl)-2-pyridinecarboxylate (7.5 g, 26.62 mmol) in 2:1 ethanol/tetrahydrofuran (300 ml) was hydrogenated with 10% palladium on carbon catalyst (1 g) for 24 hours, filtered, evaporated and dissolved in ether/1M sodium carbonate solution. The organic phase was dried (magnesium sulphate) and evaporated to give the title compound as a yellow oil (6.47 g). LC/MS: Rt=3.48 min.

### Ethyl 6-(2-{[(trifluoromethyl)sulfonyl]oxy}-1-cyclohexen-1-yl)-2-pyridinecarboxylate

Prepared by the same method as for 2-[2-[(phenylmethyl)oxy]-5-(trifluoromethyl)phenyl]-1-cyclohexen-1-yl trifluoromethanesulfonate but using ethyl 6-(2-hydroxy-1-cyclohexen-1-yl)-2-pyridinecarboxylate instead of 2-[2-[(phenylmethyl)oxy]-5-(trifluoromethyl)phenyl]cyclohexanone. LC/MS: Rt=3.54 min.

### Ethyl 6-{2-[5-bromo-2-(methyloxy)phenyl]-1-cyclohexen-1-yl}-2-pyridinecarboxylate

A mixture of Ethyl 6-(2-{[(trifluoromethyl)sulfonyl]oxy}-1-cyclohexen-1-yl)-2-pyridinecarboxylate (3.79 g, 10 mmol), 5-bromo-2-methoxyphenylboronic acid (2.54 g, 11 mmol), potassium carbonate (5.52 g, 40 mmol) and tetrakis(triphenylphosphine)palladium(0) (1.158 g, 1 mmol) in dimethoxyethane (60 ml) was stirred and heated at 80° C. under nitrogen for 2 days, 5-bromo-2-methoxyphenylboronic acid (0.51 g, 0.22 mmol) and tetrakis(triphenylphosphine)palladium(0) (150 mg, 0.13 mmol) being added after 6 hours and again after 30 hours. The resulting mixture was cooled, diluted with water/diethyl ether and the organic phase dried (magnesium sulphate) and evaporated. The residue was purified by chromatography on silica gel eluting with ethyl acetate/iso-hexane (1:9) to give the title compound as a yellow gum. (1.26 g).

LC/MS: Rt=3.91 min.

### Ethyl 6-[2-(5-bromo-2-hydroxyphenyl)-1-cyclohexen-1-yl]-2-pyridinecarboxylate

Prepared by the same method as for ethyl 6-[2-(5-chloro-2-hydroxyphenyl)-cyclohexen-1-yl]-2-pyridinecarboxylate but using ethyl 6-{2-[5-bromo-2-(methyloxy)phenyl]-1-cyclohexen-1-yl}-2-pyridinecarboxylate instead of ethyl 6-{2-[5-chloro-2-(methyloxy)phenyl]-1-cyclohexen-1-yl}-2-pyridinecarboxylate. LC/MS: Rt=3.71 min.

### Standard Alkylation Procedure

A mixture of the appropriate phenol (1 equivalent), the appropriate benzyl bromide (1.3 equivalents) and potassium carbonate (4 equivalents) in acetone (5 ml) was stirred and refluxed for 2 hours. The resulting mixture was filtered, evaporated and purified by chromatography on silica gel.

The following compounds were prepared by the standard alkylation procedure:

| COMPOUND NAME | LC/MS |
|---|---|
| Ethyl 6-{2-[2-{[(4-fluorophenyl)methyl]oxy}-5- | [M + H] 500.4, |
| (trifluoromethyl)phenyl]-1-cyclohexen-1-yl}-2- | Rt = 3.80 min |
| pyridinecarboxylate | |
| Ethyl 6-{2-[2-{[(2-fluorophenyl)methyl]oxy}-5- | [M + H] 500.4, |
| (trifluoromethyl)phenyl]-1-cyclohexen-1-yl}-2- | Rt = 4.19 min |
| pyridinecarboxylate | |
| Ethyl 6-{2-[2-{[(2,4-difluorophenyl)methyl]oxy}- | [M + H] 518.4, |
| 5-(trifluoromethyl)phenyl]-1-cyclohexen-1-yl}-2- | Rt = 3.69 min |
| pyridinecarboxylate | |
| Ethyl 6-[2-(5-(trifluoromethyl)-2-{[(2,4,5- | [M + H] 536.4, |
| trifluorophenyl)methyl]oxy}phenyl)-1- | Rt = 3.90 min |
| cyclohexen-1-yl]-2-pyridinecarboxylate | |
| Ethyl 6-{2-[2-{[(4-chloro-2- | [M + H] 534.4, |
| fluorophenyl)methyl]oxy}-5- | 536.4 |
| (trifluoromethyl)phenyl]-1-cyclohexen-1-yl}-2- | Rt = 3.92 min |
| pyridinecarboxylate | |
| Ethyl 6-[2-(5-(trifluoromethyl)-2-{[(2,4,6- | [M + H] 536.4 |
| trifluorophenyl)methyl]oxy}phenyl)-1- | Rt = 3.57 min |
| cyclohexen-1-yl]-2-pyridinecarboxylate | |
| Ethyl 6-{2-[2-{[(2-chlorophenyl)methyl]oxy}-5- | [M + H] 516.4, |
| (trifluoromethyl)phenyl]-1-cyclohexen-1-yl}-2- | 518.4 |
| pyridinecarboxylate | Rt = 3.86 min |
| Ethyl 6-{2-[2-{[(3,4-difluorophenyl)methyl]oxy}- | [M + H] 518.4 |
| 5-(trifluoromethyl)phenyl]-1-cyclohexen-1-yl}-2- | Rt = 3.97 min |
| pyridinecarboxylate | |
| Ethyl 6-{2-[2-{[(2-chloro-4- | [M + H] 534.4, |
| fluorophenyl)methyl]oxy}-5- | 536.4 |
| (trifluoromethyl)phenyl]-1-cyclohexen-1-yl}-2- | Rt = 4.18 min |
| pyridinecarboxylate | |
| Ethyl 6-{2-[2-{[(4-chlorophenyl)methyl]oxy}-5- | [M + H] 516.4, |
| (trifluoromethyl)phenyl]-1-cyclohexen-1-yl}-2- | 518.4 |
| pyridinecarboxylate | Rt = 4.27 min |
| Ethyl 6-{2-[2-[(phenylmethyl)oxy]-5- | [M + H] 482.4 |
| (trifluoromethyl)phenyl]-1-cyclohexen-1-yl}-2- | Rt = 4.02 min |
| pyridinecarboxylate | |
| Ethyl 5-{2-[2-[(phenylmethyl)oxy]-5- | [M + H] 482.4 |
| (trifluoromethyl)phenyl]-1-cyclohexen-1-yl}-3- | Rt = 3.63 min |
| pyridinecarboxylate | |
| Ethyl 5-{2-[2-{[(2-fluorophenyl)methyl]oxy}-5- | [M + H] 500.5 |
| (trifluoromethyl)phenyl]-1-cyclohexen-1-yl}-3- | Rt = 4.40 min |
| pyridinecarboxylate | |
| Ethyl 5-{2-[2-{[(2,4-difluorophenyl)methyl]oxy}- | [M + H] 518.4 |
| 5-(trifluoromethyl)phenyl]-1-cyclohexen-1-yl}-3- | Rt = 4.43 min |
| pyridinecarboxylate | |
| Ethyl 5-[2-(5-(trifluoromethyl)-2-{[(2,4,6- | [M + H] 536.4 |
| trifluorophenyl)methyl]oxy}phenyl)-1- | Rt = 4.41 min |
| cyclohexen-1-yl]-3-pyridinecarboxylate | |
| Ethyl 5-{2-[2-{[(4-fluorophenyl)methyl]oxy}-5- | [M + H] 500.5 |
| (trifluoromethyl)phenyl]-1-cyclohexen-1-yl}-3- | Rt = 4.41 min |
| pyridinecarboxylate | |
| Ethyl 5-[2-(5-(trifluoromethyl)-2-{[(2,3,4- | [M + H] 536.4 |
| trifluorophenyl)methyl]oxy}phenyl)-1- | Rt = 4.48 min |
| cyclohexen-1-yl]-3-pyridinecarboxylate | |
| Ethyl 5-[2-(5-(trifluoromethyl)-2-{[(2,4,5- | [M + H] 536.4 |
| trifluorophenyl)methyl]oxy}phenyl)-1- | Rt = 4.47 min |
| cyclohexen-1-yl]-3-pyridinecarboxylate | |
| Ethyl 5-{2-[2-{[(2-chloro-4- | [M + H] 534.4, |
| fluorophenyl)methyl]oxy}-5- | 536.4 |
| (trifluoromethyl)phenyl]-1-cyclohexen-1-yl}-3- | Rt = 4.59 min |
| pyridinecarboxylate | |
| Ethyl 5-{2-[2-{[(4-chloro-2- | [M + H] 534.4, |
| fluorophenyl)methyl]oxy}-5- | 536.4 |
| (trifluoromethyl)phenyl]-1-cyclohexen-1-yl}-3- | Rt = 4.61 min |
| pyridinecarboxylate | |
| Ethyl 6-[2-(5-chloro-2-{[(2,4,5- | [M + H] 502.3, |
| trifluorophenyl)methyl]oxy}phenyl)-1- | 502.4 |
| cyclohexen-1-yl]-2-pyridinecarboxylate | Rt = 4.41 min |
| Ethyl 6-[2-(5-chloro-2-{[(2- | [M + H] 466.4, |
| fluorophenyl)methyl]oxy}phenyl)-1-cyclohexen- | 468.4 |
| 1-yl]-2-pyridinecarboxylate | Rt = 4.28 min |
| Ethyl 6-[2-(5-chloro-2-{[(2,4,6- | [M + H] 502.3, |
| trifluorophenyl)methyl]oxy}phenyl)-1- | 504.4 |
| cyclohexen-1-yl]-2-pyridinecarboxylate | Rt = 4.19 min |
| Ethyl 6-[2-(5-chloro-2-{[(2-chloro-4- | [M + H] 500.3, |
| fluorophenyl)methyl]oxy}phenyl)-1-cyclohexen- | 503.3 |
| 1-yl]-2-pyridinecarboxylate | Rt = 4.12 min |
| Ethyl 6-[2-(5-chloro-2-{[(3,4,5- | [M + H] 502.3, |
| trifluorophenyl)methyl]oxy}phenyl)-1- | 504.4 |
| cyclohexen-1-yl]-2-pyridinecarboxylate | Rt = 3.88 min |
| Ethyl 6-[2-(5-chloro-2-{[(3,4- | [M + H] 484.4, |
| difluorophenyl)methyl]oxy}phenyl)-1- | 486.4 |
| cyclohexen-1-yl]-2-pyridinecarboxylate | Rt = 3.97 min |
| Ethyl 6-[2-(5-chloro-2-{[(4-chloro-2- | [M + H] 500.3, |
| fluorophenyl)methyl]oxy}phenyl)-1-cyclohexen- | 503.3 |
| 1-yl]-2-pyridinecarboxylate | Rt = 4.03 min |
| Ethyl 6-[2-(5-chloro-2-{[(4- | [M + H] 482.3, |
| chlorophenyl)methyl]oxy}phenyl)-1-cyclohexen- | 485.3 |
| 1-yl]-2-pyridinecarboxylate | Rt = 3.84 min |
| Ethyl 5-(2-{5-chloro-2- | [M + H] 448.4, |
| [(phenylmethyl)oxy]phenyl}-1-cyclohexen-1-yl)- | 450.4 |
| 3-pyridinecarboxylate | Rt = 4.32 min |
| Ethyl 5-[2-(5-chloro-2-{[(2- | [M + H] 466.4, |
| fluorophenyl)methyl]oxy}phenyl)-1-cyclohexen- | 468.4 |
| 1-yl]-3-pyridinecarboxylate | Rt = 4.33 min |
| Ethyl 5-[2-(5-chloro-2-{[(4- | [M + H] 466.4, |
| fluorophenyl)methyl]oxy}phenyl)-1-cyclohexen- | 468.4 |
| 1-yl]-3-pyridinecarboxylate | Rt = 4.31 min |
| Ethyl 5-[2-(5-chloro-2-{[(2,4- | [M + H] 484.4, |
| difluorophenyl)methyl]oxy}phenyl)-1- | 486.4 |
| cyclohexen-1-yl]-3-pyridinecarboxylate | Rt = 4.16 min |
| Ethyl 5-[2-(5-chloro-2-{[(2,4,5- | [M + H] 502.4, |
| trifluorophenyl)methyl]oxy}phenyl)-1- | 504.4 |
| cyclohexen-1-yl]-3-pyridinecarboxylate | Rt = 4.35 min |
| Ethyl 5-[2-(5-chloro-2-{[(2,3,4- | [M + H] 502.4, |
| trifluorophenyl)methyl]oxy}phenyl)-1- | 504.4 |
| cyclohexen-1-yl]-3-pyridinecarboxylate | Rt = 4.30 min |
| Ethyl 5-[2-(5-chloro-2-{[(2-chloro-4- | [M + H] 500.3, |
| fluorophenyl)methyl]oxy}phenyl)-1-cyclohexen- | 503.4 |
| 1-yl]-3-pyridinecarboxylate | Rt = 4.19 min |
| Ethyl 3-[2-(5-chloro-2-{[(2,4- | [M + H] 484.4, |
| difluorophenyl)methyl]oxy}phenyl)-1- | 486.4 |
| cyclohexen-1-yl]-2-pyridinecarboxylate | Rt = 4.26 min |
| Ethyl 6-(2-{5-bromo-2- | [M + H] 494.3, |
| [(phenylmethyl)oxy]phenyl}-1-cyclohexen-1-yl)- | 495.3 |
| 2-pyridinecarboxylate | Rt = 4.26 min |
| Ethyl 6-[2-(5-bromo-2-{[(2- | [M + H] 512.3, |
| fluorophenyl)methyl]oxy}phenyl)-1-cyclohexen- | 513.3 |
| 1-yl]-2-pyridinecarboxylate | Rt = 4.26 min |
| Ethyl 6-[2-(5-bromo-2-{[(4- | [M + H] 512.3, |
| fluorophenyl)methyl]oxy}phenyl)-1-cyclohexen- | 513.3 |
| 1-yl]-2-pyridinecarboxylate | Rt = 4.33 min |
| Ethyl 6-[2-(5-bromo-2-{[(2,4- | [M + H] 528.3, |
| difluorophenyl)methyl]oxy}phenyl)-1- | 531.3 |
| cyclohexen-1-yl]-2-pyridinecarboxylate | Rt = 4.37 min |
| Ethyl 6-[2-(5-bromo-2-{[(3,4- | [M + H] 530.3, |
| difluorophenyl)methyl]oxy}phenyl)-1- | 531.3 |
| cyclohexen-1-yl]-2-pyridinecarboxylate | Rt = 4.37 min |
| Ethyl 6-[2-(5-bromo-2-{[(2,3,4- | [M + H] 548.3, |
| trifluorophenyl)methyl]oxy}phenyl)-1- | 549.3 |
| cyclohexen-1-yl]-2-pyridinecarboxylate | Rt = 4.42 min |
| Ethyl 6-[2-(5-bromo-2-{[(2,4,5- | [M + H] 548.3, |
| trifluorophenyl)methyl]oxy}phenyl)-1- | 549.3 |
| cyclohexen-1-yl]-2-pyridinecarboxylate | Rt = 4.44 min |
| Ethyl 6-[2-(5-bromo-2-{[(2,4,6- | [M + H] 548.3, |
| trifluorophenyl)methyl]oxy}phenyl)-1- | 549.3 |
| cyclohexen-1-yl]-2-pyridinecarboxylate | Rt = 4.33 min |
| Ethyl 6-[2-(5-bromo-2-{[(2-chloro-4- | [M + H] 546.3, |
| fluorophenyl)methyl]oxy}phenyl)-1-cyclohexen- | 547.3 |
| 1-yl]-2-pyridinecarboxylate | Rt = 4.56 min |

### Standard Hydrolysis Procedure

The appropriate ester was dissolved in ethanol (5 ml) and 2M sodium hydroxide (2 ml) was added. After 2 hours at room temperature the ethanol was evaporated off and the residue dissolved in ethyl acetate/water. The organic phase was dried (sodium sulphate) and evaporated and the residue triturated with ether/hexane to give the sodium salt as a solid.

The following compounds were prepared by the standard hydrolysis procedure:

| EXAM- | | |
|---|---|---|
| PLE | COMPOUND NAME | LC/MS |
| 4 | Sodium 6-{2-[2-{[(4-fluorophenyl)methyl]oxy}- | [M + H] |
| | 5-(trifluoromethyl)phenyl]-1-cyclohexen-1-yl}- | 472.4 |
| | 2-pyridinecarboxylate | Rt = 3.84 |
| | | min |
| 5 | Sodium 6-{2-[2-{[(2,4- | [M + H] |
| | difluorophenyl)methyl]oxy}-5- | 490.4 |
| | (trifluoromethyl)phenyl]-1-cyclohexen-1-yl}-2- | Rt = 3.81 |
| | pyridinecarboxylate | min |
| 6 | Sodium 6-[2-(5-(trifluoromethyl)-2-{[(2,4,5- | [M + H] |
| | trifluorophenyl)methyl]oxy}phenyl)-1- | 508.4 |
| | cyclohexen-1-yl]-2-pyridinecarboxylate | Rt = 3.86 |
| | | min |
| 7 | Sodium 6-{2-[2-{[(4-chloro-2- | [M + H] |
| | fluorophenyl)methyl]oxy}-5- | 506.3, 508.2 |
| | (trifluoromethyl)phenyl]-1-cyclohexen-1-yl}-2- | Rt = 3.95 |
| | pyridinecarboxylate | min |
| 8 | Sodium 6-[2-(5-(trifluoromethyl)-2-{[(2,4,6- | [M + H] |
| | trifluorophenyl)methyl]oxy}phenyl)-1- | 508.4 |
| | cyclohexen-1-yl]-2-pyridinecarboxylate | Rt = 3.74 |
| | | min |
| 9 | Sodium 6-{2-[2-{[(2- | [M + H] |
| | chlorophenyl)methyl]oxy}-5- | 488.4, 490.4 |
| | (trifluoromethyl)phenyl]-1-cyclohexen-1-yl}-2- | Rt = 3.94 |
| | pyridinecarboxylate | min |
| 10 | Sodium 6-{2-[2-{[(3,4- | [M + H] |
| | difluorophenyl)methyl]oxy}-5- | 490.4 |
| | (trifluoromethyl)phenyl]-1-cyclohexen-1-yl}-2- | Rt = 3.74 |
| | pyridinecarboxylate | min |
| 11 | Sodium 6-{2-[2-{[(2-chloro-4- | [M + H] |
| | fluorophenyl)methyl]oxy}-5- | 506.3, 508.4 |
| | (trifluoromethyl)phenyl]-1-cyclohexen-1-yl}-2- | Rt = 3.94 |
| | pyridinecarboxylate | min |
| 12 | Sodium 6-{2-[2-{[(4- | [M + H] |
| | chlorophenyl)methyl]oxy}-5- | 488.4, 490.4 |
| | (trifluoromethyl)phenyl]-1-cyclohexen-1-yl}-2- | Rt = 3.89 |
| | pyridinecarboxylate | min |
| 13 | Sodium 6-{2-[2-{[(2-fluorophenyl)methyl]oxy}- | [M + H] |
| | 5-(trifluoromethyl)phenyl]-1-cyclohexen-1-yl}- | 472.5 |
| | 2-pyridinecarboxylate | Rt = 3.92 |
| | | min |
| 14 | Sodium 6-{2-[2-[(phenylmethyl)oxy]-5- | [M + H] |
| | (trifluoromethyl)phenyl]-1-cyclohexen-1-yl}-2- | 454.4 |
| | pyridinecarboxylate | Rt = 3.41 |
| | | min |
| 15 | Sodium 5-{2-[2-{[(2-fluorophenyl)methyl]oxy}- | [M + H] |
| | 5-(trifluoromethyl)phenyl]-1-cyclohexen-1-yl}- | 472.4 |
| | 3-pyridinecarboxylate | Rt = 4.07 |
| | | min |
| 16 | Sodium 5-{2-[2-{[(2,4- | [M + H] |
| | difluorophenyl)methyl]oxy}-5- | 490.4 |
| | (trifluoromethyl)phenyl]-1-cyclohexen-1-yl}-3- | Rt = 4.10 |
| | pyridinecarboxylate | min |
| 17 | Sodium 5-[2-(5-(trifluoromethyl)-2-{[(2,4,6- | [M + H] |
| | trifluorophenyl)methyl]oxy}phenyl)-1- | 508.4 |
| | cyclohexen-1-yl]-3-pyridinecarboxylate | Rt = 4.08 |
| | | min |
| 18 | Sodium 5-{2-[2-{[(4-fluorophenyl)methyl]oxy}- | [M + H] |
| | 5-(trifluoromethyl)phenyl]-1-cyclohexen-1-yl}- | 472.4 |
| | 3-pyridinecarboxylate | Rt = 4.09 |
| | | min |
| 19 | Sodium 5-[2-(5-(trifluoromethyl)-2-{[(2,3,4- | [M + H] |
| | trifluorophenyl)methyl]oxy}phenyl)-1- | 508.4 |
| | cyclohexen-1-yl]-3-pyridinecarboxylate | Rt = 4.17 |
| | | min |
| 20 | Sodium 5-[2-(5-(trifluoromethyl)-2-{[(2,4,5- | [M + H] |
| | trifluorophenyl)methyl]oxy}phenyl)-1- | 508.4 |
| | cyclohexen-1-yl]-3-pyridinecarboxylate | Rt = 4.16 |
| | | min |
| 21 | Sodium 5-{2-[2-{[(2-chloro-4- | [M + H] |
| | fluorophenyl)methyl]oxy}-5- | 506.3, 508.4 |
| | (trifluoromethyl)phenyl]-1-cyclohexen-1-yl}-3- | Rt = 4.31 |
| | pyridinecarboxylate | min |
| 22 | Sodium 5-{2-[2-{[(4-chloro-2- | [M + H] |
| | fluorophenyl)methyl]oxy}-5- | 506.3, 508.4 |
| | (trifluoromethyl)phenyl]-1-cyclohexen-1-yl}-3- | Rt = 4.31 |
| | pyridinecarboxylate | min |
| 23 | Sodium 5-{2-[2-[(phenylmethyl)oxy]-5- | [M + H] |
| | (trifluoromethyl)phenyl]-1-cyclohexen-1-yl}-3- | 454.4 |
| | pyridinecarboxylate | Rt = 3.87 |
| | | min |
| 24 | Sodium 6-[2-(5-chloro-2-{[{2,4,5- | [M + H] |
| | trifluorophenyl)methyl]oxy}phenyl)-1- | 474.4, 476.4 |
| | cyclohexen-1-yl]-2-pyridinecarboxylate | Rt = 3.93 |
| | | min |
| 25 | Sodium 6-[2-(5-chloro-2-{[(2- | [M + H] |
| | fluorophenyl)methyl]oxy}phenyl)-1- | 438.4, 440.4 |
| | cyclohexen-1-yl]-2-pyridinecarboxylate | Rt = 3.62 |
| | | min |
| 26 | Sodium 6-[2-(5-chloro-2-{[(2,4,6- | [M + H] |
| | trifluorophenyl)methyl]oxy}phenyl)-1- | 474.4, 476.4 |
| | cyclohexen-1-yl]-2-pyridinecarboxylate | Rt = 3.47 |
| | | min |
| 27 | Sodium 6-[2-(5-chloro-2-{[(2-chloro-4- | [M + H] |
| | fluorophenyl)methyl]oxy}phenyl)-1- | 472.3, 475.3 |
| | cyclohexen-1-yl]-2-pyridinecarboxylate | Rt = 3.92 |
| | | min |
| 28 | Sodium 6-[2-(5-chloro-2-{[(3,4,5- | [M + H] |
| | trifluorophenyl)methyl]oxy}phenyl)-1- | 474.4, 476.4 |
| | cyclohexen-1-yl]-2-pyridinecarboxylate | Rt = 3.69 |
| | | min |
| 29 | Sodium 6-[2-(5-chloro-2-{[(3,4- | [M + H] |
| | difluorophenyl)methyl]oxy}phenyl)-1- | 456.4, 458.4 |
| | cyclohexen-1-yl]-2-pyridinecarboxylate | Rt = 3.62 |
| | | min |
| 30 | Sodium 6-[2-(5-chloro-2-{[(4-chloro-2- | [M + H] |
| | fluorophenyl)methyl]oxy}phenyl)-1- | 472.3, 475.3 |
| | cyclohexen-1-yl]-2-pyridinecarboxylate | Rt = 3.84 |
| | | min |
| 31 | Sodium 6-[2-(5-chloro-2-{[(4- | [M + H] |
| | chlorophenyl)methyl]oxy}phenyl)-1- | 454.4, 457.4 |
| | cyclohexen-1-yl]-2-pyridinecarboxylate | Rt = 3.80 |
| | | min |
| 32 | Sodium 5-(2-{5-chloro-2- | [M + H] |
| | [(phenylmethyl)oxy]phenyl}-1-cyclohexen-1- | 420.5, 422.5 |
| | yl)-3-pyridinecarboxylate | Rt = 3.89 |
| | | min |
| 33 | Sodium 5-[2-(5-chloro-2-{[(2- | [M + H] |
| | fluorophenyl)methyl]oxy}phenyl)-1- | 438.5, 440.5 |
| | cyclohexen-1-yl]-3-pyridinecarboxylate | Rt = 3.86 |
| | | min |
| 34 | Sodium 5-[2-(5-chloro-2-{[(4- | [M + H] |
| | fluorophenyl)methyl]oxy}phenyl)-1- | 438.2, 440.2 |
| | cyclohexen-1-yl]-3-pyridinecarboxylate | Rt = 4.06 |
| | | min |
| 35 | Sodium 5-[2-(5-chloro-2-{[(2,4- | [M + H] |
| | difluorophenyl)methyl]oxy}phenyl)-1- | 456.2, 458.2 |
| | cyclohexen-1-yl]-3-pyridinecarboxylate | Rt = 4.09 |
| | | min |
| 36 | Sodium 5-[2-(5-chloro-2-{[(2,4,5- | [M + H] |
| | trifluorophenyl)methyl]oxy}phenyl)-1- | 474.4, 476.5 |
| | cyclohexen-1-yl]-3-pyridinecarboxylate | Rt = 4.18 |
| | | min |
| 37 | Sodium 5-[2-(5-chloro-2-{[(2,3,4- | [M + H] |
| | trifluorophenyl)methyl]oxy}phenyl)-1- | 474.4, 476.5 |
| | cyclohexen-1-yl]-3-pyridinecarboxylate | Rt = 4.19 |
| | | min |
| 38 | Sodium 5-[2-(5-chloro-2-{[(2-chloro-4- | [M + H] |
| | fluorophenyl)methyl]oxy}phenyl)-1- | 454.4, 457.4 |
| | cyclohexen-1-yl]-3-pyridinecarboxylate | Rt = 3.80 |
| | | min |
| 39 | Sodium 6-(2-{5-bromo-2- | [M + H] |
| | [(phenylmethyl)oxy]phenyl}-1-cyclohexen-1 - | 466.3, 467.4 |
| | yl)-2-pyridinecarboxylate | Rt = 3.93 |
| | | min |
| 40 | Sodium 6-[2-(5-bromo-2-{[(2- | [M + H] |
| | fluorophenyl)methyl]oxy}phenyl)-1- | 484.3, 485.3 |
| | cyclohexen-1-yl]-2-pyridinecarboxylate | Rt = 3.97 |
| | | min |
| 41 | Sodium 6-[2-(5-bromo-2-{[(4- | [M + H] |
| | fluorophenyl)methyl]oxy}phenyl)-1- | 482.3, 485.3 |
| | cyclohexen-1-yl]-2-pyridinecarboxylate | Rt = 3.95 |
| | | min |
| 42 | Sodium 6-[2-(5-bromo-2-{[(2,4- | [M + H] |
| | difluorophenyl)methyl]oxy}phenyl)-1- | 502.3, 503.3 |
| | cyclohexen-1-yl]-2-pyridinecarboxylate | Rt = 4.01 |
| | | min |
| 43 | Sodium 6-[2-(5-bromo-2-{[(3,4- | [M + H] |
| | difluorophenyl)methyl]oxy}phenyl)-1- | 502.3, 503.3 |
| | cyclohexen-1-yl]-2-pyridinecarboxylate | Rt = 4.01 |
| | | min |
| 44 | Sodium 6-[2-(5-bromo-2-{[(2,3,4- | [M + H] |
| | trifluorophenyl)methyl]oxy}phenyl)-1- | 520.2, 521.3 |
| | cyclohexen-1-yl]-2-pyridinecarboxylate | Rt = 4.07 |
| | | min |
| 45 | Sodium 6-[2-(5-bromo-2-{[(2,4,5- | [M + H] |
| | trifluorophenyl)methyl]oxy}phenyl)-1- | 520.2, 521.3 |
| | cyclohexen-1-yl]-2-pyridinecarboxylate | Rt = 4.08 |
| | | min |
| 46 | Sodium 6-[2-(5-bromo-2-{[(2,4,6- | [M + H] |
| | trifluorophenyl)methyl]oxy}phenyl)-1- | 520.3, 521.3 |
| | cyclohexen-1-yl]-2-pyridinecarboxylate | Rt = 3.97 |
| | | min |
| 47 | Sodium 6-[2-(5-bromo-2-{[(2-chloro-4- | [M + H] |
| | fluorophenyl)methyl]oxy}phenyl)-1- | 518.2, 520.2 |
| | cyclohexen-1-yl]-2-pyridinecarboxylate | Rt = 4.27 |
| | | min |
| 48 | Sodium 3-[2-(5-chloro-2-{[(2,4- | [M + H] |
| | difluorophenyl)methyl]oxy}phenyl)-1- | 456.3, 458.3 |
| | cyclohexen-1-yl]-2-pyridinecarboxylate | Rt = 4.06 |
| | | min |

It is to be understood that the present invention covers all combinations of particular and preferred subgroups described herein above.

### Assays for Determining Biological Activity

The compounds of formula (I) can be tested using the following assays to demonstrate their prostanoid antagonist or agonist activity in vitro and in vivo and their selectivity. The prostaglandin receptors investigated are DP, EP₁, EP₂, EP₃, EP₄, FP, IP and TP.

The ability of compounds to antagonise EP₁ & EP₃ receptors may be demonstrated using a functional calcium mobilisation assay. Briefly, the antagonist properties of compounds are assessed by their ability to inhibit the mobilisation of intracellular calcium ([Ca²⁺]ᵢ) in response to activation of EP₁ or EP₃ receptors by the natural agonist hormone prostaglandin E₂ (PGE₂). Increasing concentrations of antagonist reduce the amount of calcium that a given concentration of PGE₂ can mobilise. The net effect is to displace the PGE₂ concentration-effect curve to higher concentrations of PGE₂. The amount of calcium produced is assessed using a calcium-sensitive fluorescent dye such as Fluo-3, AM and a suitable instrument such as a Fluorimetric Imaging Plate Reader (FLIPR). Increasing amounts of [Ca²⁺]ᵢ produced by receptor activation increase the amount of fluorescence produced by the dye and give rise to an increasing signal. The signal may be detected using the FLIPR instrument and the data generated may be analysed with suitable curve-fitting software.

The human EP₁ or EP₃ calcium mobilisation assay (hereafter referred to as ‘the calcium assay’) utilises Chinese hamster ovary-K1 (CHO-K1) cells into which a stable vector containing either EP₁ or EP₃ cDNA has previously been transfected. Cells are cultured in suitable flasks containing culture medium such as DMEM:F-12 supplemented with 10% v/v foetal calf serum, 2 mM L-glutamine, 0.25 mg/ml geneticin and 10 μg/ml puromycin.

For assay, cells are harvested using a proprietary reagent that dislodges cells such as Versene. Cells are re-suspended in a suitable quantity of fresh culture media for introduction into a 384-well plate. Following incubation for 24 hours at 37° C. the culture media is replaced with a medium containing fluo-3 and the detergent pluronic acid, and a further incubation takes place. Concentrations of compounds are then added to the plate in order to construct concentration-effect curves. This may be performed on the FLIPR in order to assess the agonist properties of the compounds. Concentrations of PGE₂ are then added to the plate in order to assess the antagonist properties of the compounds.

The data so generated may be analysed by means of a computerised curve-fitting routine. The concentration of compound that elicits a half-maximal inhibition of the calcium mobilisation induced by PGE₂ (pIC₅₀) may then be estimated.

### Binding Assay for the Human Prostanoid EP₁ Receptor

Competition assay using [³H]-PGE₂.

Compound potencies are determined using a radioligand binding assay. In this assay compound potencies are determined from their ability to compete with tritiated prostaglandin E₂ ([³H]-PGE₂) for binding to the human EP₁ receptor.

This assay utilises Chinese hamster ovary-K1 (CHO-K1) cells into which a stable vector containing the EP₁ cDNA has previously been transfected. Cells are cultured in suitable flasks containing culture medium such as DMEM:F-12 supplemented with 10% v/v foetal calf serum, 2 mM L-glutamine, 0.25 mg/ml geneticin, 10 μg/ml puromycin and 10 μM indomethacin.

Cells are detached from the culture flasks by incubation in calcium and magnesium free phosphate buffered saline containing 1 mM disodium ethylenediaminetetraacetic acid (Na₂EDTA) and 10 μM indomethacin for 5 min. The cells are isolated by centrifugation at 250×g for 5 mins and suspended in an ice cold buffer such as 50 mM Tris, 1 mM Na₂EDTA, 140 mM NaCl, 10 μM indomethacin (pH 7.4). The cells are homogenised using a Polytron tissue disrupter (2×10s burst at full setting), centrifuged at 48,000×g for 20 mins and the pellet containing the membrane fraction is washed three times by suspension and centrifugation at 48,000×g for 20 mins. The final membrane pellet is suspended in an assay buffer such as 10 mM 2-[N-morpholino]ethanesulphonic acid, 1 mM Na₂EDTA, 10 mM MgCl₂ (pH 6). Aliquots are frozen at −80° C. until required.

For the binding assay the cell membranes, competing compounds and [³H]-PGE₂ (3 nM final assay concentration) are incubated in a final volume of 100 μl for 30 min at 30° C. All reagents are prepared in assay buffer. Reactions are terminated by rapid vacuum filtration over GF/B filters using a Brandell cell harvester. The filters are washed with ice cold assay buffer, dried and the radioactivity retained on the filters is measured by liquid scintillation counting in Packard TopCount scintillation counter.

The data are analysed using non linear curve fitting techniques (GraphPad Prism 3) to determine the concentration of compound producing 50% inhibition of specific binding (IC₅₀).

By application of these techniques, compounds of the examples had an antagonist pIC₅₀ value of between 6.5 and 9.5 at EP₁ receptors and pIC50 value of <6.0 at EP₃ receptors.

No toxicological effects are indicated/expected when a compound (of the invention) is administered in the above mentioned dosage range.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation the following claims:

What is claimed is:

1. A compound of formula (I): 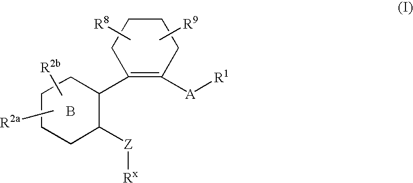
wherein:
A represents an optionally substituted aryl, or an optionally substituted 5- or 6-membered heterocyclyl ring, or an optionally substituted bicyclic heterocyclyl group;
B represents a phenyl or pyridyl ring;
Z represents O, S, SO, or SO₂;
R¹ represents CO₂H, CN, CONR⁵R⁶, CH₂CO₂H, optionally substituted SO₂alkyl, SO₂NR⁵R⁶, NR⁵CONR⁵R⁶, COalkyl, 2H-tetrazol-5-yl-methyl, optionally substituted bicyclic heterocycle or optionally substituted heterocyclyl;
R^{2a} and R^{2b} each independently represents hydrogen, halo, optionally substituted alkyl, optionally substituted alkoxy, CN, SO₂alkyl, SR⁵, NO₂, optionally substituted aryl, CONR⁵R⁶ or optionally substituted heteroaryl;
R^{x} represents optionally substituted alkyl wherein 1 or 2 of the non-terminal carbon atoms are optionally substituted by a group independently selected from NR⁴, O and SOₙ, wherein n is 0, 1 or 2, optionally substituted alkenyl or optionally substituted alkynyl: or R^{x} represents optionally substituted CQ^{a}Q^{b} heterocyclyl, optionally substituted CQ^{a}Q^{b}-bicyclic heterocyclyl or optionally substituted CQ^{a}Q^{b}-aryl;
R⁴ represents hydrogen or an optionally substituted alkyl;
R⁵ represents hydrogen or an optionally substituted alkyl;
R⁶ represents hydrogen or optionally substituted alkyl, optionally substituted heteroaryl, optionally substituted SO₂aryl, optionally substituted SO₂alkyl, optionally substituted SO₂heteroaryl, CN, optionally substituted CQ^{a}Q^{b} aryl, optionally substituted CQ^{a}Q^{b} heteroaryl or COR⁷;
R⁷ represents hydrogen, optionally substituted alkyl, optionally substituted heteroaryl or optionally substituted aryl;
R⁸ and R⁹ each independently represents hydrogen, chloro, fluoro, CF₃, C₁₋₃alkoxy or C₁₋₃alkyl;
Q^{a} and Q^{b} each independently selected from hydrogen and CH₃; and
when A is a 6-membered ring the R¹ substituent and cyclohexene ring are attached to carbon atoms 1,2-, 1,3- or 1,4-relative to each other, and when A is a five-membered ring or bicyclic heterocyclyl group the R¹ substituent and cyclohexene ring are attached to substitutable carbon atoms 1,2- or 1,3-relative to each other,
or a derivatives thereof.

2. A compound according to claim 1 wherein A is pyridyl.

3. A compound according to claim 1 wherein R¹ represents CO₂H.

4. A compound selected from:
6-[2-(5-chloro-2-{[(4-fluorophenyl)methyl]oxy}phenyl)-1-cyclohexen-1-yl]-2-pyridinecarboxylic acid;
6-[2-(5-chloro-2-{[(2,4-difluorophenyl)methyl]oxy}phenyl)-1-cyclohexen-1-yl]-2-pyridinecarboxylic acid;
6-[2-(5-chloro-2-{[(2,4-difluorophenyl)methyl]oxy}phenyl)-1-cyclohexen-1-yl]-2-pyridinecarboxylic acid;
6-{2-[2-{[(4-fluorophenyl)methyl]oxy}-5-(trifluoromethyl)phenyl]-1-cyclohexen-1-yl}-2-pyridinecarboxylic acid;
6-{2-[2-{[(2,4-difluorophenyl)methyl]oxy}-5-(trifluoromethyl)phenyl]-1-cyclohexen-1-yl}-2-pyridinecarboxylic acid;
6-[2-(5-(trifluoromethyl)-2-[(2,4,5-trifluorophenyl)methyl]oxy phenyl)-1-cyclohexen-1-yl]-2-pyridinecarboxylic acid;
6-{2-[2-{[(4-chloro-2-fluorophenyl)methyl]oxy}-5-(trifluoromethyl)phenyl]-1-cyclohexen-1-yl}-2-pyridinecarboxylic acid;
6-[2-(5-(trifluoromethyl)-2-{[(2,4,6-trifluorophenyl)methyl]oxy}phenyl)-1-cyclohexen-1-yl]-2-pyridinecarboxylic acid;
6-{2-[2-{[(2-chlorophenyl)methyl]oxy}-5-(trifluoromethyl)phenyl]-1-cyclohexen-1-yl}-2-pyridinecarboxylic acid;
6-{2-[2-{[(3,4-difluorophenyl)methyl]oxy}-5-(trifluoromethyl)phenyl]-1-cyclohexen-1-yl}-2-pyridinecarboxylic acid;
6-(2-[2-{[(2-chloro-4-fluorophenyl)methyl]oxy)-5-(trifluoromethyl)phenyl]-1-cyclohexen-1-yl}-2-pyridinecarboxylic acid;
6-{2-[2-{[(4-chlorophenyl)methyl]oxy}-5-(trifluoromethyl)phenyl]-1-cyclohexen-1-yl}-2-pyridinecarboxylic acid;
6-{2-[2-{[(2-fluorophenyl)methyl]oxy}-5-(trifluoromethyl)phenyl]-1-cyclohexen-1-yl}-2-pyridinecarboxylic acid;
6-{2-[2-[(phenylmethyl)oxy]-5-(trifluoromethyl)phenyl]-1-cyclohexen-1-yl}-2-pyridinecarboxylic acid;
5-{2-[2-{[(2-fluorophenyl)methyl]oxy}-5-(trifluoromethyl)phenyl]-1-cyclohexen-1-yl}-3-pyridinecarboxylic acid;
5-{2-[2-{[(2,4-difluorophenyl)methyl]oxy}-5-(trifluoromethyl)phenyl]-1-cyclohexen-1-yl}-3-pyridinecarboxylic acid;
5-[2-(5-(trifluoromethyl)-2-{([(2,4,6-trifluorophenyl)methyl]oxy}phenyl)-1-cyclohexen-1-yl]-3-pyridinecarboxylic acid;
5-{2-[2-{[(4-fluorophenyl)methyl]oxy}-5-(trifluoromethyl)phenyl]-1-cyclohexen-1-yl}-3-pyridinecarboxylic acid;
5-[2-(5-(trifluoromethyl)-2-{[(2,3,4-trifluorophenyl)methyl]oxy}phenyl)-1-cyclohexen-1-yl]-3-pyridinecarboxylic acid;
5-[2-(5-(trifluoromethyl)-2-{[(2,4,5-trifluorophenyl)methyl]oxy}phenyl)-1-cyclohexen-1-yl]-3-pyridinecarboxylic acid;
5-{2-[2-{[(2-chloro-4-fluorophenyl)methyl]oxy}-5-(trifluoromethyl)phenyl]-1-cyclohexen-1-yl}-3-pyridinecarboxylic acid;
5-{2-[2-{[(4-chloro-2-fluorophenyl)methyl]oxy}-5-(trifluoromethyl)phenyl]-1-cyclohexen-1-yl}-3-pyridinecarboxylic acid;
5-{2-[2-[(phenylmethyl)oxy]-5-(trifluoromethyl)phenyl]-1-cyclohexen-1-yl}-3-pyridinecarboxylic acid;
6-[2-(5-chloro-2-{[(2,4,5-trifluorophenyl)methyl]oxy}phenyl)-1-cyclohexen-1-yl]-2-pyridinecarboxylic acid;
6-[2-(5-chloro-2-{[(2-fluorophenyl)methyl]oxy}phenyl)-1-cyclohexen-1-yl]-2-pyridinecarboxylic acid;
6-[2-(5-chloro-2-{([(2,4,6-trifluorophenyl)methyl]oxy}phenyl)-1-cyclohexen-1-yl]-2-pyridinecarboxylic acid;
6-[2-(5-chloro-2-{[(2-chloro-4-fluorophenyl)methyl]oxy}phenyl)-1-cyclohexen-1-yl]-2-pyridinecarboxylic acid;
6-[2-(5-chloro-2-{[(3,4,5-trifluorophenyl)methyl]oxy}phenyl)-1-cyclohexen-1-yl]-2-pyridinecarboxylic acid;
6-[2-(5-chloro-2-{[(3,4-difluorophenyl)methyl]oxy}phenyl)-1-cyclohexen-1-yl]-2-pyridinecarboxylic acid;
6-[2-(5-chloro-2-{[(4-chloro-2-fluorophenyl)methyl]oxy}phenyl)-1-cyclohexen-1-yl]-2-pyridinecarboxylic acid;
6-[2-(5-chloro-2-{[(4-chlorophenyl)methyl]oxy}phenyl)-1-cyclohexen-1-yl]-2-pyridinecarboxylic acid;
5-(2-{5-chloro-2-[(phenylmethyl)oxy]phenyl}-1-cyclohexen-1-yl)-3-pyridinecarboxylate
5-[2-(5-chloro-2-{[(2-fluorophenyl)methyl]oxy}phenyl)-1-cyclohexen-1-yl]-3-pyridinecarboxylic acid;
5-[2-(5-chloro-2-{[(4-fluorophenyl)methyl]oxy}phenyl)-1-cyclohexen-1-yl]-3-pyridinecarboxylic acid;
5-[2-(5-chloro-2-{[(2,4-difluorophenyl)methyl]oxy}phenyl)-1-cyclohexen-1-yl]-3-pyridinecarboxylic acid;
5-[2-(5-chloro-2-{[(2,4,5-trifluorophenyl)methyl]oxy}phenyl)-1-cyclohexen-1-yl]-3-pyridinecarboxylic acid;
5-[2-(5-chloro-2-{[(2,3,4-trifluorophenyl)methyl]oxy}phenyl)-1-cyclohexen-1-yl]-3-pyridinecarboxylic acid;
5-[2-(5-chloro-2-{[(2-chloro-4-fluorophenyl)methyl]oxy}phenyl)-1-cyclohexen-1-yl]-3-pyridinecarboxylic acid;
6-(2-{5-bromo-2-[(phenylmethyl)oxy]phenyl}-1-cyclohexen-1-yl)-2-pyridinecarboxylic acid;
6-[2-(5-bromo-2-{[(2-fluorophenyl)methyl]oxy}phenyl)-1-cyclohexen-1-yl]-2-pyridinecarboxylic acid;
6-[2-(5-bromo-2-{[(4-fluorophenyl)methyl]oxy}phenyl)-1-cyclohexen-1-yl]-2-pyridinecarboxylic acid;
6-[2-(5-bromo-2-{[(2,4-difluorophenyl)methyl]oxy}phenyl)-1-cyclohexen-1-yl]-2-pyridinecarboxylic acid;
6-[2-(5-bromo-2-{[(3,4-difluorophenyl)methyl]oxy}phenyl)-1-cyclohexen-1-yl]-2-pyridinecarboxylic acid;
6-[2-(5-bromo-2-{[(2,3,4-trifluorophenyl)methyl]oxy}phenyl)-1-cyclohexen-1-yl]-2-pyridinecarboxylic acid;
6-[2-(5-bromo-2-{[(2,4,5-trifluorophenyl)methyl]oxy}phenyl)-1-cyclohexen-1-yl]-2-pyridinecarboxylic acid;
6-[2-(5-bromo-2-{[(2,4,6-trifluorophenyl)methyl]oxy}phenyl)-1-cyclohexen-1-yl]-2-pyridinecarboxylic acid;
6-[2-(5-bromo-2-{[(2-chloro-4-fluorophenyl)methyl]oxy}phenyl)-1-cyclohexen-1-yl]-2-pyridinecarboxylic acid; and
3-[2-(5-chloro-2-{[(2,4-difluorophenyl)methyl]oxy}phenyl)-1-cyclohexen-1-yl]-2-pyridinecarboxylic acid;
and derivatives thereof.

5. A pharmaceutical composition comprising a compound according to claim 1 or a pharmaceutically acceptable derivative thereof together with a pharmaceutical carrier and/or excipient.

6.-7. (canceled)

8. A method of treating a human or animal subject suffering from a condition which is mediated by the action of PGE₂ at EP₁ receptors which comprises administering to said subject an effective amount of a compound according to claim 1 or a pharmaceutically acceptable derivative thereof.

9. A method of treating a human or animal subject suffering from a pain, inflammatory, immunological, bone, neurodegenerative or renal disorder, which method comprises administering to said subject an effective amount of a compound according to claim 1 or a pharmaceutically acceptable derivative thereof.

10. A method of treating a human or animal subject suffering from inflammatory pain, neuropathic pain or visceral pain which method comprises administering to said subject an effective amount of a compound according to claim 1 or a pharmaceutically acceptable derivative thereof.

11.-13. (canceled)

14. The method of claim 8, wherein the subject is a human.

15. The method of claim 9, wherein the subject is a human.

16. The method of claim 10, wherein the subject is a human.

17. A method of mediating EP1 receptors, comprising the step of administering an effective amount of a compound according to claim 1 or a pharmaceutically acceptable derivative thereof.
